(19) United States
(12) Patent Application Publication
(10) Pub. No.: US 2012/0164022 A1
(43) Pub. Date: Jun. 28, 2012

(54) METHODS AND DEVICES FOR PROCESSING OBJECTS BY APPLYING ELECTROMAGNETIC (EM) ENERGY

(75) Inventors: Ginat MUGINSTEIN, Evern Yehuda (IL); Daniel Selinger, Tel-Aviv (IL); Eyal Torres, Savyon (IL); Ohad Karnielli, Kiryat Tivon (IL)

(73) Assignee: Goji Limited

(21) Appl. No.: 13/334,981

(22) Filed: Dec. 22, 2011

Related U.S. Application Data
(60) Provisional application No. 61/425,874, filed on Dec. 22, 2010.
(60) Provisional application No. 61/466,545, filed on Mar. 23, 2011.
(60) Provisional application No. 61/521,082, filed on Aug. 8, 2011.

(51) Int. Cl. A61L 2/08; G21K 5/00; B09C 1/00

(52) U.S. Cl. 422/22; 422/105; 405/128.7; 250/492.1

(57) ABSTRACT

An apparatus is disclosed for applying radio frequency (RF) energy to an object in an energy application zone via at least one radiating element. The apparatus is selected from a group consisting of sterilizers, pasteurizers, drying cabinets, sintering furnaces, curing furnaces, soil remediation apparatuses, smelting furnaces, melting furnaces and plasma generators. The apparatus comprises at least one processor configured to determine a value indicative of energy absorbable by the object at least one of or each of a plurality of MSEs; and cause RF energy to be supplied to the at least one radiating element in at least a subset of the plurality of MSEs, wherein energy supplied to the at least one radiating element at each of the subset of MSEs is a function of the value indicative of energy absorbable at each MSE.

### RELATED APPLICATIONS

This application is based upon and claims the benefit of priority of: U.S. Provisional Patent Application No. 61/425,874 filed Dec. 22, 2010; U.S. Provisional Patent Application No. 61/466,545 filed Mar. 23, 2011; and U.S. Provisional Patent Application No. 61/521,082 filed Aug. 8, 2011, the contents of each of which is incorporated herein by reference.

### TECHNICAL FIELD

This application relates to devices and methods for applying electromagnetic energy (EM) energy (e.g., EM energy from a source emitting EM radiation in the Radio Frequency range, hereinafter abbreviated as “RF energy”) for processing an object.

### BACKGROUND

Electromagnetic waves have been used in various applications to supply EM energy to objects. In the case of radio frequency (RF) radiation for example, EM energy may be supplied using a magnetron, which is typically tuned to a single frequency for supplying EM energy only in that frequency. One example of a commonly used device for supplying EM energy is a microwave oven. Typical microwave ovens supply EM energy at or about a single frequency of 2.45 GHz.

### SUMMARY OF EXEMPLARY ASPECTS OF THE DISCLOSURE

Exemplary aspects of the invention include apparatuses and methods for applying EM energy to an object in an energy application zone.

Exemplary aspects of the invention may be directed to some apparatuses and methods for applying EM energy to an object in an energy application zone via at least one radiating element. The apparatuses may be selected from a group including: cooking appliances, drying cabinets, sintering furnaces, curing furnaces, soil remediation apparatuses, smelting furnaces, melting furnaces, sterilizers, pasteurizers, food drying apparatuses, and plasma generators. The object may be selected from a group including: food items to be cooked, pet foods to be cooked or dried, polymers to be cured, powders to be sintered, soil to be remediated, objects to be dried, metallic ores to be smelted, metal to be melted, and gas to be ionized in order to, among other things, generate plasma.

According to some exemplary aspects, one or more apparatuses or methods may involve determining a value indicative of energy absorbable by the object at least one of or each of a plurality of MSEs. This may occur, for example, through the use of a controller, which may be further configured to cause energy to be supplied to at least one radiating element in at least a subset of the plurality of MSEs. Energy applied to the zone at each of the subset of MSEs may be a function of the value indicative of energy absorbable at each MSE. Alternatively or additionally, energy applied to the zone at each of the subset of MSEs may be a function of the value indicative of energy absorbable at more than one of the plurality of MSEs.

According to some exemplary aspects of the disclosure, one or more apparatuses or methods may include determining a value indicative of energy absorbable by an object at least one of or each of a plurality of MSEs, and causing energy to be supplied to at least one radiating element in at least a subset of the plurality of MSEs to an energy application zone. Energy applied to the zone at each of the subset of MSEs may be inversely related to the value indicative of energy absorbable at each MSE.

In yet other aspects, one or more apparatuses or methods may adjust energy supplied to the radiating element(s) as a function of the MSE at which the energy is absorbed.

Alternatively, or additionally, exemplary apparatuses and methods may determine a desired energy absorption level in the object to be processed (e.g., heated) at least one of or each of a plurality of MSEs, and may adjust energy applied at each MSE in order to, for example, target the desired energy absorption level to the object to be processed at each MSE.

Exemplary aspects of the invention include an apparatus for applying radio frequency (RF) energy to an object in an energy application zone via at least one radiating element, wherein the apparatus is selected from a group consisting of: sterilizers, pasteurizers, drying cabinets, sintering furnaces, curing furnaces, soil remediation apparatuses, smelting furnaces, melting furnaces and plasma generators, the apparatus comprising at least one processor configured to determine a value indicative of energy absorbable by the object at least one of or each of a plurality of MSEs, and cause RF energy to be supplied to the at least one radiating element in at least a subset of the plurality of MSEs, wherein energy supplied to the at least one radiating element at each of the subset of MSEs is a function of the value indicative of energy absorbable at each MSE. The object may be selected from a group consisting of: pressed powders, metallic powders, ceramic powders, MMCs, pet food, metallic ores, parts to be sterilized, plasma to be generated, soil, curable polymers and dryable objects. The apparatus may further comprise a source of electromagnetic (EM) energy for supplying the RF energy to the at least one radiating element. The apparatus may further comprise at least one radiating element. The apparatus may further comprise a cavity, wherein the energy application zone is within the cavity. The apparatus may further comprise a system for applying a protective atmosphere to the apparatus. The apparatus may further comprise a conveyor configured to convey object to the apparatus. The apparatus may further comprise at least one sensor configured to monitor a temperature of the object. The processor may be further configured to adjust the application of RF energy based on the monitored temperature. The apparatus may further comprise at least one sensor configured to monitor a moisture level of the object. The processor may be further configured to adjust the application of RF energy based to the monitored moisture. The apparatus may further comprise at least one sensor configured to monitor contamination in the object. The processor may be further configured to adjust the application of RF energy based to the monitored contamination. The apparatus may further comprise a convection heating system. The processor may be further configured to control the convection heating system. The processor may be further configured to cause the at least radiating element to apply energy to the object in an amount sufficient to heat at least a portion of the object. The processor may be further configured to cause substantially uniform energy dissipation in at least a selected portion of the object at a plurality of locations of the object in the energy application zone. The processor may be further configured to cause substantially uniform energy dissipation in the object at a plurality of locations of the object in the zone. The value indicative of energy absorbable at each MSE may be a dissipation ratio at the corresponding MSE. Exemplary aspects of the invention include an apparatus for applying electromagnetic energy (EM) energy to an object in an energy application zone via at least one radiating element, wherein the apparatus is selected from a group consisting of: sterilizers, pasteurizers, drying cabinets, sintering furnaces, curing furnaces, soil remediation apparatuses, smelting furnaces, melting furnaces and plasma generators, the apparatus comprising at least one processor configured to determine a value indicative of energy absorbable by the object at at least one of or each of a plurality of MSEs, and cause energy to be supplied to the at least one radiating element in at least a subset of the plurality of MSEs, wherein the energy supplied to the at least one radiating element at each of the subset of MSEs is inversely related to the value indicative of energy absorbable at each MSE.

Exemplary aspects of the invention include an apparatus for applying electromagnetic energy (EM) energy to an object in an energy application zone via at least one radiating element, wherein the apparatus is selected from a group consisting of: sterilizers, pasteurizers, drying cabinets, sintering furnaces, curing furnaces, soil remediation apparatuses, smelting furnaces, melting furnaces and plasma generators, the apparatus comprising at least one processor configured to determine a desired energy absorption amount at least one of or each of a plurality of MSEs, and adjust energy supplied to the at least one radiating element at each of the plurality of MSEs in order to target the desired energy absorption amount.

Exemplary aspects of the invention include a method for applying electromagnetic (EM) energy to an object, wherein the object is selected from a group consisting of: sterilized, pasteurized, or other pet food polymer, pressed powder, soil, metallic ores, metal, and gas, the method comprising controlling a source of electromagnetic EM energy in order to supply EM energy at a plurality of MSEs to at least one radiating element, determining a value indicative of energy absorbable by the object at each of the plurality of MSEs, and adjusting an amount of EM energy applied at each of the plurality of MSEs based on the value indicative of energy absorbable at each MSE to at least one of cook the sterilized or pasteurized pet food, dry the pet food, cure the polymer, sinter the pressed powder, remediate the soil, smelt the metallic ore, melt the metal, or ionize the gas.

Exemplary aspects of the invention include a method of sterilizing at least one portion of an object using radiofrequency (RF) energy comprising controlling application of RF energy to the at least one portion object, selecting at least one modulation space element (MSE) that causes at least one portion of the object to receive energy sufficient to sterilize the portion of the object, and applying energy at the selected MSE space element to the object for a time sufficient to sterilize the portion of the object. The object may be dry. Applying energy at the selected MSE may heat the portion of the object to a desired sterilizing temperature. The object may be chosen from food items, food utensils, fabrics, and medical devices. The object may be chosen from food items having a moisture content less than 50 wt %, relative to the total weight of the at least one item. The object may comprise metal. The object may comprise at least one dielectric material. The at least one dielectric material is a coating. The method may further comprise determining a value indicative of RF energy absorbable in the object at a plurality of MSEs, and applying more energy over MSEs of the plurality of MSEs that are associated with lower values of the value indicative of RF energy absorbable than over MSE, of the plurality of SEs associated with higher values of the value indicative of RF energy absorbable.

The drawings and detailed description which follow contain numerous alternative examples consistent with the invention. A summary of every feature disclosed is beyond the object of this summary section. For a more detailed description of exemplary aspects of the invention, reference should be made to the drawings, detailed description, and claims, which are incorporated into this summary by reference.

### BRIEF DESCRIPTION OF THE DRAWINGS

FIG. 1 is a diagrammatic representation of an apparatus for applying EM energy to an object, in accordance with some exemplary embodiments of the present invention;

FIG. 2 is a view of a cavity, in accordance with some exemplary embodiments of the present invention;

FIG. 3 is a view of a cavity, in accordance with some exemplary embodiments of the present invention;

FIG.

FIG. 4 is a diagrammatic representation of an apparatus for applying EM energy to an object, in accordance with some exemplary embodiments of the present invention;

FIG. 5 is a flow chart of a method for applying EM energy to an energy application zone in accordance with some embodiments of the present invention;

FIG. 6 is a flow chart of a process for curing bulk parts made from polymer in accordance with some embodiments of the present invention;

FIG. 7 is a flow chart of a process for curing thin polymeric layers in accordance with some embodiments of the present invention;

FIG. 8 is a flow chart of a process for rapid prototyping of three dimensional parts made from polymer layers in accordance with some embodiments of the present invention;

FIG. 9 is a diagrammatic presentation of an RF furnace in accordance with some embodiments of the present invention;

FIG. 10 is a diagrammatic presentation of an RF furnace that including a plurality of shelves in accordance with some embodiments of the present invention;

FIG. 11 is a diagrammatic presentation of an RF furnace including a plurality of floating shelves in accordance with some embodiments of the present invention;

FIG. 12 is a flow chart of a process for sintering object in an RF sintering furnace in accordance with some embodiments of the present invention;

FIG. 13 is a flow chart of a drying process of an object in an RF drying cabinet in accordance with some embodiments of the present invention;

FIG. 14 is a flow chart of a process of ores in an RF smelting furnace in accordance with some embodiments of the present invention;

FIG. 15 is a flow chart of a melting process of metals and alloys in an RF melting furnace in accordance with some embodiments of the present invention;

FIG. 16 is a flow chart of a process for remediation of contaminated soil in a close batch RF applicator in accordance with some embodiments of the present invention;

FIG. 17 is a flowchart illustrating a continuous process for remediation of contaminated soil in an open batch RF applicator in accordance with some embodiments of the invention;

FIG. 18 is a flow chart of a method for cooking and/or preparing a pet food by applying RF energy, in accordance with some embodiments of the present invention; and

FIG. 19 is a flow chart of a method for sterilizing or pasteurizing an object by applying RF energy, in accordance with some embodiments of the present invention.

### DETAILED DESCRIPTION

Reference will now be made in detail to exemplary embodiments of the invention, examples of which are illustrated in the accompanying drawings. When appropriate, the same reference numbers are used throughout the drawings to refer to the same or like parts.

In one respect, the invention may involve apparatus and methods for applying EM energy. The term EM energy, as used herein, includes energy deliverable by electromagnetic radiation in all or portions of the electromagnetic spectrum, including but not limited to, radio frequency (RF), infrared (IR), near infrared, visible light, ultraviolet, etc. In one particular example, applied EM energy may include RF energy with a wavelength in free space of 100 km to 1 mm, which corresponds to a frequency of 3 KHz to 300 GHz, respectively. In some other examples, the applied EM energy may fall within frequency bands between 500 MHz to 1500 MHz or between 700 MHz to 1200 MHz or between 800 MHz-1 GHz. Applying energy in the RF portion of the electromagnetic spectrum is referred herein as applying RF energy. Microwave and ultra high frequency (UHF) energy, for example, are both within the RF range. In some other examples, the applied EM energy may fall only within one or more ISM frequency bands, for example, between 433.05 and 434.79 MHz, between 902 and 928 MHz, between 2400 and 2500 MHz, and/or between 5725 and 5875 MHz. Even though examples of the invention are described herein in connection with the application of RF energy, these descriptions are provided to illustrate a few exemplary principles of the invention, and are not intended to limit the invention to any particular portion of the electromagnetic spectrum.

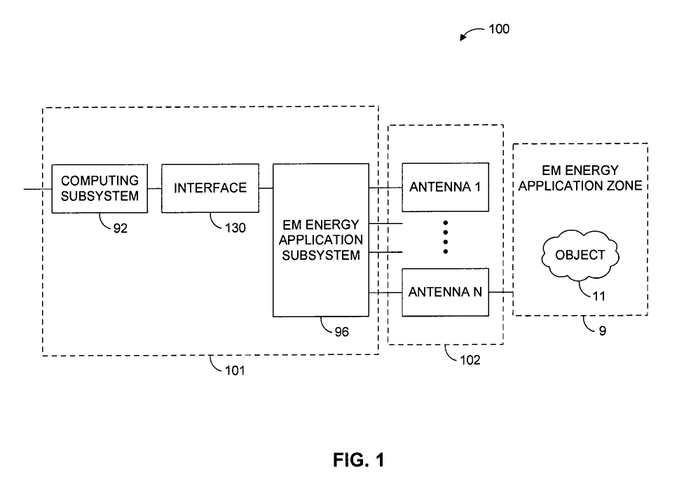

In certain embodiments, the application of EM energy may occur in an “energy application zone”, such as energy application zone 9, as shown in FIG. 1. Energy application zone 9 may include any void, location, region, or area where EM energy may be applied. It may be hollow, or may be filled or partially filled with liquids, solids, gases, or combinations thereof. By way of example only, energy application zone 9 may include an interior of an enclosure, interior of a partial enclosure, open space, solid, or partial solid that allows existence, propagation, and/or resonance of electromagnetic waves. Zone 9 may include a conveyor belt or a rotating plate. For purposes of this disclosure, all such energy application zones may alternatively be referred to as cavities. It is to be understood that an object is considered “in” the energy application zone if at least a portion of the object is located in the zone or if some portion of the object receives delivered electromagnetic radiation.

In accordance with some embodiments of the invention, an apparatus or method may involve the use of at least one source configured to supply EM energy to radiating element(s) to be applied to the energy application zone. A “source” may include any component(s) that are suitable for generating and supplying EM energy. Consistent with some embodiments of the invention, EM energy may be applied to the energy application zone in the form of propagating electromagnetic waves at predetermined wavelengths or frequencies (also known as electromagnetic radiation). As used consistently herein, “propagating electromagnetic waves” may include resonating waves, evanescent waves, and waves that travel through a medium in any other manner. Electromagnetic radiation carries energy that may be imparted to (or dissipated into) matter with which it interacts.

In certain embodiments, EM energy may be applied to an object 11. References to an “object” (or “object to be heated” or “object to be processed”) to which EM energy is applied is not limited to a particular form. An object may include a liquid, semi-liquid, solid, semi-solid, or gas, depending upon the particular process with which the invention is utilized. The object may also include composites or mixtures of matter in differing phases. Thus, by way of non-limiting example, the term “object” encompasses such matter as food to be defrosted or cooked; clothes or other wet material to be dried; frozen organs to be thawed; chemicals to be reacted; fuel or other combustible material to be combusted; hydrated material to be dehydrated, gases to be expanded; liquids to be heated, boiled or vaporized, or any other material for which there is a desire to apply, even nominally, EM energy.

In some embodiments, a portion of EM energy applied to energy application zone 9 may be absorbed by object 11. In some embodiments, another portion of the EM energy supplied or applied to energy application zone 9 may be absorbed by various elements (e.g., food residue, particle residue, additional objects, structures associated with zone 9, or any other EM energy-absorbing materials found in zone 9) associated with energy application zone 9. Energy application zone 9 may also include loss constituents that do not, themselves, absorb an appreciable amount of EM energy, but otherwise account for EM energy losses. Such loss constitutes may include, for example, cracks, seams, joints, doors, an interface between a door and a cavity, or any other loss mechanisms associated with energy application zone 9.

FIG. 1 is a diagrammatic representation of an apparatus 100 for applying EM energy to an object. Apparatus 100 may include a controller 101, an array of radiating elements, for example antennas 102 illustrated in FIG. 1, including one or more radiating elements, and energy application zone 9. Controller 101 may be electrically coupled to one or more antennas 102. As used herein, the term “electrically coupled” refers to one or more either direct or indirect electrical connections. Controller 101 may include a computing subsystem 92, an interface 130, and an EM energy application subsystem 96. Based on an output of computing subsystem 92, energy application subsystem 96 may respond by generating one or more radio frequency signals to be supplied to antennas 102. In turn, the one or more antennas 102 may radiate EM energy into energy application zone 9. In certain embodiments, this energy can interact with object 11 positioned within energy application zone 9.

Consistent with the presently disclosed embodiments, computing subsystem 92 may include a general purpose or special purpose computer. Computing subsystem 92 may be configured to generate control signals for controlling EM energy application subsystem 96 via interface 130. Computing subsystem 92 may further receive measured signals from EM energy application subsystem 96 via interface 130.

While controller 101 is illustrated for exemplary purposes as having three subcomponents, control functions may be consolidated in fewer components, or additional components may be included consistent with the desired function and/or design of a particular embodiment.

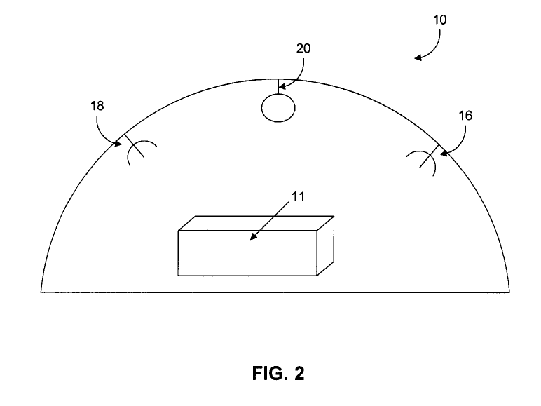

Exemplary energy application zone 9 may include locations where energy is applied in an oven, chamber, tank, dryer, thawer, sterilizers, pasteurizers, food drying apparatuses, dehydrator, reactor, engine, chemical or biological processing apparatus, furnace, incinerator, material shaping or forming apparatus, conveyor, combustion zone, cooler, freezer, etc. Thus, consistent with the presently disclosed embodiments, energy application zone 9 may include an electromagnetic resonator 10 (also known as cavity resonator, or cavity) (illustrated for example in FIG. 2). At times, energy application zone 9 may be congruent with the object or a portion of the object (e.g., the object or a portion thereof, is or may define the energy application zone).

FIG. 2 shows a sectional view of a cavity 10, which is one exemplary embodiment of energy application zone 9. Cavity 10 may be cylindrical in shape (or any other suitable shape, such as semi-cylindrical, rectangular, elliptical, cuboid, symmetrical, asymmetrical, irregular, and regular, among others) and may be made of a conductor, such as aluminum, stainless steel or any suitable metal or other conductive material. In some embodiments, cavity 10 may include walls coated and/or covered with a protective coating, for example, made from materials transparent to EM energy, e.g., metallic oxides or others. In some embodiments, cavity 10 may have a spherical shape or hemispherical shape (for example as illustrated in FIG. 2). Cavity 10 may be resonant in a predetermined range of frequencies (e.g., within the UHF or microwave range of frequencies, such as between 300 MHz and 3 GHz, or between 400 MHz and 1 GHZ). It is also contemplated that cavity 10 may be closed, e.g., completely enclosed (e.g., by conductor materials), bounded at least partially, or open, e.g., having non-bounded openings. The general methodology of the invention is not limited to any particular cavity shape or configuration, as discussed earlier. FIG. 2 shows a sensor 20 and antennas 16 and 18 (examples of antennas 102 shown in FIG. 1).

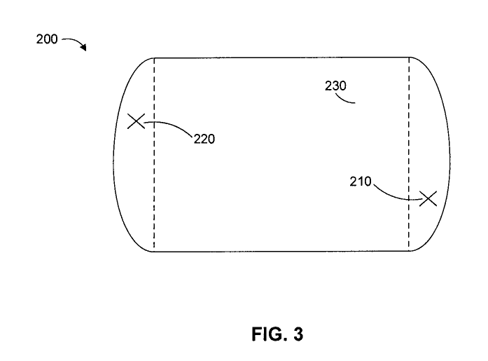

FIG. 3 shows a top sectional view of a cavity 200 according to another exemplary embodiment of energy application zone 9. FIG. 3 shows antennas 210 and 220 (as examples of antennas 102 shown in FIG. 1). Cavity 200 comprises a space 230 for receiving object 11 (not shown). Space 230, as shown between the dotted lines in FIG. 3, has an essentially rectangular cross section, which may be adapted for receiving a tray on top of which object 11 may be placed.

In some embodiments, field adjusting element(s) (not illustrated) may be provided in energy application zone 9, for example, in cavity 10 and/or cavity 200. Field adjusting element(s) may be adjusted to change the electromagnetic wave pattern in the cavity in a way that selectively directs the EM energy from one or more of antennas 16 and 18 (or 210 and 220) into object 11. Additionally or alternatively, field adjusting element(s) may be further adjusted to simultaneously match at least one of the antennas that act as transmitters, and thus reduce coupling to the other antennas that act as receivers.

Additionally, one or more sensor(s) (or detector(s)) 20 may be used to sense (or detect) information (e.g., signals) relating to object 11 and/or to the energy application process and/or the energy application zone. At times, one or more antennas, e.g., antenna 16, 18, 210 or 220, may be used as sensors. The sensors may be used to sense any information, including electromagnetic power, temperature, weight, humidity, motion, etc. The sensed information may be used for any purpose, including process verification, automation, authentication, safety, etc.

In the presently disclosed embodiments, more than one feed and/or a plurality of radiating elements (e.g., antennas) may be provided. The radiating elements may be located on one or more surfaces of, e.g., an enclosure defining the energy application zone. Alternatively, radiating elements may be located inside or outside the energy application zone. One or more of the radiating elements may be near to, in contact with, in the vicinity of or even embedded in object 11 (e.g., when the object is a liquid). The orientation and/or configuration of each radiating element may be distinct or the same, based on the specific energy application, e.g., based on a desired target effect. Each radiating element may be positioned, adjusted, and/or oriented to transmit electromagnetic waves along a same direction, or various different directions. Furthermore, the location, orientation, and configuration of each radiating element may be predetermined before applying energy to the object. Alternatively or additionally, the location, orientation, and configuration of each radiating element may be dynamically adjusted, for example, by using a processor, during operation of the apparatus and/or between rounds of energy application. The invention is not limited to radiating elements having particular structures or locations within the apparatus.

As represented by the block diagram of FIG. 1, apparatus 100 may include at least one radiating element in the form of at least one antenna 102 for delivery of EM energy to energy application zone 9. One or more of the antenna(s) may also be configured to receive EM energy from energy application zone 9. In other words, an antenna, as used herein may function as a transmitter, a receiver, or both, depending on a particular application and configuration. When an antenna acts as a receiver of EM energy from an energy application zone (e.g., reflected electromagnetic waves), the antenna receives EM energy from the energy application zone.

As used herein, the terms “radiating element” and “antenna” may broadly refer to any structure from which EM energy may radiate (emitted) and/or be received, regardless of whether the structure was originally designed for the purposes of radiating or receiving energy, and regardless of whether the structure serves any additional function. For example, a radiating element or an antenna may include an aperture/slot antenna, or an antenna which includes a plurality of terminals transmitting in unison, either at the same time or at a controlled dynamic phase difference (e.g., a phased array antenna). Consistent with some exemplary embodiments, antennas 102 may include an EM energy transmitter (referred to herein as “a transmitting antenna”) that feeds (applies) energy into EM energy application zone 9, an EM energy receiver (referred herein as “a receiving antenna”) that receives energy from zone 9, or a combination of both a transmitter and a receiver. For example, a first antenna may be configured to apply EM energy to zone 9, and a second antenna may be configured to receive energy from the first antenna. In some embodiments, one or more antennas may each serve as both receivers and transmitters. In some embodiments, one or more antennas may serve a dual function while one or more other antennas may serve a single function. So, for example, a single antenna may be configured to both deliver EM energy to the zone 9 and to receive EM energy via the zone 9; a first antenna may be configured to deliver EM energy to the zone 9, and a second antenna may be configured to receive EM energy via the zone 9; or a plurality of antennas could be used, where at least one of the plurality of antennas may be configured to both deliver EM energy to zone 9 and to receive EM energy via zone 9. At times, in addition to or as an alternative to delivering and/or receiving energy, an antenna may also be adjusted to affect the field pattern. For example, various properties of the antenna, such as position, location, orientation, temperature, etc., may be adjusted. Different antenna property settings may result in differing electromagnetic field patterns within the energy application zone thereby affecting energy absorption in the object. Therefore, antenna adjustments may constitute one or more variables that can be varied in an energy delivery scheme.

Consistent with the presently disclosed embodiments, energy may be supplied and/or provided to one or more transmitting antennas. Energy supplied to a transmitting antenna may result in energy emitted by the transmitting antenna (or transmitting radiating element) (referred to herein as “incident energy”). The incident energy may be applied to zone 9, and may be in an amount equal to an amount of energy supplied to the transmitting antenna(s) by a source. A portion of the incident energy may be dissipated in the object or absorbed by the object (referred to herein as “dissipated energy” or “absorbed energy”). Another portion may be reflected back to the transmitting antenna (referred to herein as “reflected energy”). Reflected energy may include, for example, energy reflected back to the transmitting antenna due to mismatch caused by the object and/or the energy application zone, e.g., impedance mismatch. Reflected energy may also include energy retained by the port of the transmitting antenna (e.g., energy that is emitted by the antenna but does not flow into the zone). The rest of the incident energy, other than the reflected energy and dissipated energy may be coupled to one or more receiving antennas (receiving radiating elements) other than the transmitting antenna (referred to herein as “coupled energy.”). Therefore, the incident energy (“I”) supplied to the transmitting antenna may include all of the dissipated energy (“D”), reflected energy (“R”), and coupled energy (“T”), and may be expressed according to the relationship:

I=D+R+ΣTᵢ.

In accordance with certain aspects of the invention, the one or more transmitting antennas may deliver EM energy into zone 9. Energy delivered by a transmitting antenna into the zone (referred to herein as “delivered energy” or (d)) may be the incident energy emitted by the antenna minus the reflected energy at the same antenna. That is, the delivered energy may be the net energy that flows from the transmitting antenna to the zone, i.e., d=I−R. Alternatively, the delivered energy may also be represented as the sum of dissipated energy and coupled energy, i.e., d=D+T (where T=ΣTi).

In certain embodiments, the application of EM energy may occur via one or more power feeds. A feed may include one or more waveguides and/or one or more radiating elements (e.g., antennas 102) for applying EM energy to the zone. Such antennas may include, for example, patch antennas, fractal antennas, helix antennas, log-periodic antennas, spiral antennas, slot antennas, dipole antennas, loop antennas, slow wave antennas, leaky wave antennas or any other structures capable of transmitting and/or receiving EM energy.

The invention is not limited to antennas having particular structures or locations. Antennas, e.g., antenna 102, may be polarized in differing directions in order to, for example, reduce coupling, enhance specific field pattern(s), increase the energy delivery efficiency and support and/or enable a specific algorithm(s). The foregoing are examples only, and polarization may be used for other purposes as well. In one example, three antennas may be placed parallel to orthogonal coordinates. However, it is contemplated that any suitable number of antennas (such as one, two, three, four, five, six, seven, eight, etc.) may be used. For example, a higher number of antennas may add flexibility in system design and improve control of energy distribution, e.g., greater uniformity and/or resolution of energy application in zone 9.

In some embodiments, one or more slow wave antenna(s) may be provided in the energy application zone either in addition to or as an alternative to radiating element(s) such as antenna(s) 102. A slow-wave antenna may refer to a wave-guiding structure that possesses a mechanism that permits it to emit power along all or part of its length. The slow wave antenna may comprise a plurality of slots to enable EM energy to be emitted. In some embodiments, the object to be processed, e.g., cooked, may be placed in the energy application zone so that a coupling may be formed between an evanescent EM wave (e.g., emitted from a slow wave antenna) and the object. An evanescent EM wave in free space (e.g., in the vicinity of the slow wave antenna) may be non-evanescent in the object.

Antennas, e.g., antenna 102, may be configured to feed energy at specifically chosen modulation space elements, referred to herein as MSEs, which are optionally chosen by controller 101. The term “modulation space” or “MS” is used to collectively refer to all the parameters that may affect a field pattern in the energy application zone and all combinations thereof. In some embodiments, the “MS” may include all possible components that may be used and their potential settings (absolute and/or relative to others) and adjustable parameters associated with the components. For example, the “MS” may include a plurality of variable parameters, the number of antennas, their positioning and/or orientation (if modifiable), the useable bandwidth, a set of all useable frequencies and any combinations thereof, power settings, phases, etc. The MS may have any number of possible variable parameters, ranging between one parameter only (e.g., a one dimensional MS limited to frequency only or phase only—or other single parameter), two or more dimensions (e.g., varying frequency and amplitude or varying frequency and phase together within the same MS), or many more.

Each variable parameter associated with the MS is referred to as an MS dimension. By way of example, three dimensions modulation space may include a frequency (F), phase (P), and amplitude (A). That is frequency, phase, and amplitude (e.g., an amplitude difference between two or more waves being emitted at the same time) of the electromagnetic waves are modulated during energy delivery, while all the other parameters may be predetermined and fixed during energy delivery. The modulation space is depicted in three dimensions for ease of discussion only. The MS may have any number of dimensions, e.g., one dimension, two dimensions, four dimensions, n dimensions, etc. In one example, a one dimensional modulation space oven may provide MSEs that differ one from the other only by frequency.

The term “modulation space element” or “MSE,” may refer to a specific set of values of the variable parameters in MS. Therefore, the MS may also be considered to be a collection of all possible MSEs. For example, two MSEs may differ one from another in the relative amplitudes of the energy being supplied to a plurality of radiating elements. For example, in three-dimensional MS. MSE has a specific frequency F(i), a specific phase P(i), and a specific amplitude A(i). If even one of these MSE variables changes, then the new set defines another MSE. For example, (3 GHz, 30°, 12 V) and (3 GHz, 60°, 12 V) are two different MSEs, although only the phase component is different.

Differing combinations of these MS parameters will lead to differing field patterns across the energy application zone and differing energy distribution patterns in the object. A plurality of MSEs that can be executed sequentially or simultaneously to excite a particular field pattern in the energy application zone may be collectively referred to as an “energy delivery scheme.” For example, an energy delivery scheme may consist of three MSEs: (F(1), P(1), A(1)); (F(2), P(2), A(2)) (F(3), P(3), A(3)). Such an energy application scheme may result in applying the first, second, and third MSE to the energy application zone.

The invention, in its broadest sense, is not limited to any particular number of MSEs or MSE combinations. Various MSE combinations may be used depending on the requirements of a particular application and/or on a desired energy transfer profile, and/or given equipment, e.g., cavity dimensions. The number of options that may be employed could be as few as two or as many as the designer desires, depending on factors such as intended use, level of desired control, hardware or software resolution and cost.

In certain embodiments, there may be provided at least one processor. As used herein, the term “processor” may include an electric circuit that performs a logic operation on input or inputs. For example, such a processor may include one or more integrated circuits, microchips, microcontrollers, microprocessors, all or part of a central processing unit (CPU), graphics processing unit (GPU), digital signal processors (DSP), field-programmable gate array (FPGA) or other circuit suitable for executing instructions or performing logic operations. The at least one processor may be coincident with or may be part of controller 101.

The instructions executed by the processor may, for example, be pre-loaded into the processor or may be stored in a separate memory unit such as a RAM, a ROM, a hard disk, an optical disk, a magnetic medium, a flash memory, other permanent, fixed, or volatile memory, or any other mechanism capable of storing instructions for the processor. The processor(s) may be customized for a particular use, or can be configured for general-purpose use and can perform different functions by executing different software.

If more than one processor is employed, all may be of similar construction, or they may be of differing constructions electrically connected or disconnected from each other. They may be separate circuits or integrated in a single circuit. When more than one processor is used, they may be configured to operate independently or collaboratively. They may be coupled electrically, magnetically, optically, acoustically, mechanically or by other means permitting them to interact.

The at least one processor may be configured to cause EM energy to be applied to zone 9 via one or more radiating elements (e.g., antennas), for example across a series of MSEs, in order to apply EM energy at each such MSE to an object 11. For example, the at least one processor may be configured to regulate one or more components of controller 101 in order to cause the energy to be applied.

In certain embodiments, the at least one processor may be configured to determine a value indicative of energy absorbable by the object at each of a plurality of MSEs. This may occur, for example, using one or more lookup tables, by pre-programming the processor or memory associated with the processor, and/or by testing an object in an energy application zone to determine its absorbable energy characteristics. One exemplary way to conduct such a test is through a sweep.

As used herein, a sweep may include, for example, the transmission over time of energy at more than one MSE. For example, a sweep may include the sequential transmission of energy at multiple MSEs in one or more contiguous MSE band; the sequential transmission of energy at multiple MSEs in more than one non-contiguous MSE band; the sequential transmission of energy at individual non-contiguous MSEs; and/or the transmission of synthesized pulses having a desired MSE/power spectral content (e.g., a synthesized pulse in time). The MSE bands may be contiguous or non-contiguous. Thus, during an MSE sweeping process, the at least one processor may regulate the energy supplied to the at least one antenna to sequentially apply (transmit) EM energy at various MSEs to zone 9, and to receive feedback which serves as an indicator of the energy absorbable by object 11. While the invention is not limited to any particular measure of feedback indicative of energy absorption in the object, various exemplary indicative values are discussed below.

During the sweeping process, EM energy application subsystem 96 may be regulated to receive EM energy reflected and/or coupled at antenna(s) 102, and to communicate the measured energy information (e.g., information pertaining to and/or related to and/or associated with the measured energy) back to computing subsystem 92 via interface 130, as illustrated in FIG. 1. Computing subsystem 92 may then be regulated to determine a value indicative of energy absorbable by object 11 at each of a plurality of MSEs based on the received information. Consistent with some of the presently disclosed embodiments, a value indicative of the absorbable energy may include a dissipation ratio (referred to herein as “DR”) associated with each of a plurality of MSEs. As referred to herein, a “dissipation ratio” (or “absorption efficiency” or “power efficiency”), may be defined as a ratio between EM energy absorbed by object 11 and EM energy supplied into the transmitting radiating element. As referred to herein, a “dissipation ratio” (or “absorption efficiency” or “power efficiency”), may be defined as a ratio between EM energy absorbed by object 11 and EM energy delivered to the energy application zone.

Energy that may be dissipated or absorbed by an object is referred to herein as “absorbable energy” or “absorbed energy” or dissipated energy. Absorbable energy may be an indicator of the object's capacity to absorb energy or the ability of the apparatus to cause energy to dissipate in a given object (for example—an indication of the upper limit thereof). In some of the presently disclosed embodiments, absorbable energy may be calculated as a product of the incident energy (e.g., maximum incident energy) supplied to the at least one antenna and the dissipation ratio. Reflected energy (e.g., the energy not absorbed or coupled) may, for example, be a value indicative of energy absorbed by the object. By way of another example, a processor might calculate or estimate absorbable energy based on the portion of the incident energy that is reflected and the portion that is coupled. That estimate or calculation may serve as a value indicative of absorbed and/or absorbable energy.

During an MSE sweep, for example, the at least one processor may be configured to control a source of EM energy such that energy is sequentially supplied to an object at a series of MSEs. The at least one processor might then receive a signal indicative of energy reflected at each MSE and, optionally, also a signal indicative of the energy coupled to other antennas at each MSE. Using a known amount of incident energy supplied to the antenna and a known amount of energy reflected and/or coupled (e.g., thereby indicating an amount of energy absorbed at each MSE), an absorbable energy indicator may be calculated or estimated. Alternatively, the processor might simply rely on an indicator of reflection and/or coupling as a value indicative of absorbable energy.

Absorbable energy may also include energy that may be dissipated by the structures of the energy application zone in which the object is located (e.g., cavity walls) or leakage of energy at an interface between an oven cavity and an oven door. Because absorption in metallic or conducting material (e.g., the cavity walls or elements within the cavity) is characterized by a large quality factor (also known as a “Q factor”), MSEs having a large Q factor may be identified as being associated with conducting material, and at times, a choice may be made not to transmit energy in such MSEs. In that case, the amount of EM energy absorbed in the cavity walls may be substantially small, and thus, the amount of EM energy absorbed in the object may be substantially equal to the amount of absorbable energy.

In some of the presently disclosed embodiments, a dissipation ratio may be calculated using formula (1):

DR=A/S (1)

wherein S is the incident energy supplied to a transmitting radiating element and A is the energy absorbed in the object. Both S and A may be calculated by integrating over time, power detected by power detectors (e.g., detector 2040). For t=ti, wherein ti may be any moment in time during which energy is applied to the energy application zone, equation (1) may have the form:

DR=PA/PS; (1*)

Wherein PA is the power absorbed and Ps the power supplied (the incident power). PA may be evaluated using equation (2):

PA=Ps−Pout; (2)

wherein Pout stands for the power detected by all the detectors (e.g., receiving radiating elements), denoted as Pdetect(i) in the ith detector, in and around the energy application zone, when Ps was supplied (applied) by a radiating element at a certain MSE, see equation (3):

Pout=ΣPdetect(i) (3)

If the only available detectors are the one associated with the radiating elements, DR may be calculated using three detected power parameters PS, PR and PC and equation (1*) may have the form of equation (4):

DR=(PS−PR−PC)/PS (4)

where PS represents the EM energy and/or power supplied into zone 9 by transmitting radiating elements (e.g., 102, 16 or 18), PR represents the EM energy and/or power reflected/returned to the transmitting radiating element, and PC represents the EM energy coupled to the other radiating elements function as receiving radiating elements. DR may be a value between 0 and 1, and thus may be represented by a percentage number.

For example, consistent with an embodiment which is designed for three radiating elements controller 101 or processor 2030 may be configured to determine the input reflection coefficients S₁₁, S₂₂, and S₃₃ and transfer coefficients S₁₂, S₂₁, S₁₃, S₃₁, S₂₃, and S₃₂ based on measured power and/or energy information during the sweep. Accordingly, the dissipation ratio DR corresponding to radiating element 1 may be calculated based on the above mentioned reflection and transmission coefficients, according to equation (5):

DR1=1−(IS₁₁I²+IS₁₂I²+IS₁₃I²). (5)

As shown in equation (5), the dissipation ratio may be different at different radiating elements. Thus, in some embodiments, amount of energy supplied to a particular radiating element may be determined based on the dissipation ratio associated with that particular radiating element.

During the EM energy application, additional parameters may be calculated and monitored based on the dissipation ratio. In some embodiments, an average dissipation ratio, for example averaged over all applied MSEs, may be calculated, optionally for each radiating element.

In some embodiments, the average DR may be calculated over time for each MSE.

In certain embodiments, controller 101 may configured to determine an RF energy application protocol by adjusting the amount of RF energy supplied at each MSE based on a value indicative of EM energy absorbable in the object by sweeping over a plurality of MSEs. For example controller 101 may use the dissipation ratio calculated for each DR(MSEi) to determine the amount of energy to be supplied at each MSEi as a function of the DR(MSEi). In some embodiments, the energy applied at MSEi may be inversely related to the DR(MSEi). Such an inverse relationship may be applied to other values indicative of energy absorbable and may involve a general trend. For example, when the value indicative of absorbable energy in a particular MSE subset (i.e., one or more MSEs) tends to be relatively high, the actual incident energy at that MSE subset may be relatively low. When an indicator of absorbable energy in a particular MSE subset tends to be relatively low, the incident energy may be relatively high. This substantially inverse relationship may be even more closely correlated. For example, the applied energy may be set such that its product with the value indicative of energy absorbable (i.e., the absorbable energy by object 11) is substantially constant across the MSEs applied. In other embodiments, other relations may be applied, for example a constant amount of energy may be applied at least a sub-group of MSEs.

Another value indicative of EM energy absorbable in the object according to the invention may be the complex input impedance of a radiating element, denoted herein as Zin, its real part, denoted Real(Zin), and/or its imaginary part, denoted Img(Zin). The controller may associate each of the Real(Zin) and Img(Zin) values measured one each of the radiating elements when RF energy was applied to the energy application zone at a particular MSE. The controller (e.g., controller 101 or processor 2030) may further be configured to control the application of RF energy at each MSE based on the measured Real(Zin) and/or Img(Zin). For example the resonance nature of an EM field excited in the energy application zone at a particular MSE from a plurality of MSEs may be determined based on the value of Img(Zin) at that particular MSE. For example, in some embodiments, the resonance character may be different at MSEs for which Img(Zin) is 0 than for MSEs for which Img(Zin) is not zero.

Some exemplary energy delivery schemes may lead to more spatially uniform energy absorption in the object. As used herein, “spatial uniformity” may refer to a condition where the absorbed energy across the object or a portion (e.g., a selected portion) of the object that is targeted for energy application is substantially constant (for example per volume unit or per mass unit). In some embodiments, the energy absorption is considered “substantially constant” if the variation of the dissipated energy at different locations of the object is lower than a threshold value. For instance, a deviation may be calculated based on the distribution of the dissipated energy in the object, and the absorbable energy is considered “substantially constant” if the deviation between the dissipation values of different parts of the object is less than 50%. Because in many cases spatially uniform energy absorption may result in spatially uniform temperature increase, consistent with the presently disclosed embodiments, “spatial uniformity” may also refer to a condition where the temperature increase across the object or a portion of the object that is targeted for energy application is substantially constant. The temperature increase may be measured by a sensing device, for example a temperature sensor provided in zone 9. In some embodiments, spatial uniformity may be defined as a condition, where a given property of the object is uniform or substantially uniform after processing, e.g., after a heating process. Examples of such properties may include temperature, readiness degree (e.g., of food cooked in the oven), mean particle size (e.g., in a sintering process), etc.

In order to control energy application to an object (e.g., to control an amount of energy applied at each MSE), controller 101 may be configured to hold substantially constant the amount of time at which energy is supplied to antennas 102 at each MSE, while varying the amount of power supplied at each MSE as a function of the value indicative of energy absorbable. In some embodiments, controller 101 may be configured to cause the energy to be supplied to the antenna at a particular MSE or MSEs at a power level substantially equal to a maximum power level of the device and/or an amplifier at the respective MSE(s).

Alternatively or additionally, controller 101 may be configured to vary the period of time during which energy is applied at each MSE as a function of the value indicative of energy absorbable. At times, both the duration and power at which each MSE is applied are varied as a function of the value indicative of energy absorbable. Varying the power and/or duration of energy supplied at each MSE may be used to cause substantially uniform energy absorption in the object or to have a controlled spatial pattern of energy absorption, for example, based on feedback from the dissipation properties of the object at each applied MSE.

Consistent with some other embodiments, controller 101 may be configured to cause the source to supply no energy at all at particular MSE(s). Similarly, if the value indicative of energy absorbable exceeds a predetermined threshold, controller 101 may be configured to cause the antenna to apply energy at a power level less than a maximum power level of the antenna.

Because absorbable energy can change based on a host of factors including object temperature, in some embodiments, it may be beneficial to regularly update absorbable energy values and adjust energy application based on the updated absorption values. These updates can occur multiple times a second, or can occur every few seconds or longer, depending on the requirements of a particular application.

In accordance with an aspect of some embodiments of the invention, the at least one processor (e.g., controller 101 or processor 2030) may be configured to determine a desired and/or target energy absorption level at each of a plurality of MSEs and adjust energy supplied to the antenna at each MSE in order to obtain the target energy absorption level at each MSE. For example, controller 101 may be configured to target a desired energy absorption level at each MSE in order to achieve or approximate substantially uniform energy absorption across a range of MSEs.

Alternatively, controller 101 may be configured to provide a target energy absorption level at each of a plurality of object portions, which collectively may be referred to as an energy absorption profile across the object. An absorption profile may include uniform energy absorption in the object, non-uniform energy absorption in the object, differing energy absorption values in differing portions of the object, substantially uniform absorption in one or more portions of the object, or any other desirable pattern of energy absorption in an object or portion(s) of an object.

In some embodiments, the at least one processor may be configured to adjust energy supplied from the antenna at each MSE in order to obtain a desired target energy effect and/or energy effect in the object, for example: a different amount of energy may be provided to different parts and/or regions of the object.

In some embodiments, a resolution of the different regions (for example, to which different amounts of energy are applied) and/or a resolution of a discretization of the zone (e.g., the zone may be divided into a plurality of regions) may be a fraction of the wavelength of the applied EM energy, e.g., on the order of λ/10, λ/5, λ/2. For example, for 900 MHz, the corresponding wavelength (λ) in air (∈=1) is 33.3 cm and the resolution may be on the order of 3 cm, e.g., (3 cm)³ resolution. In water, for example, the wavelength is approximately 9 times shorter at the same frequency (900 MHz), thus the resolution may be in the order of 0.33 cm, e.g., (0.33 cm)³. In meat, for example, the wavelength corresponding to frequency of 900 MHz is about 7 times shorter than in air and the resolution may be in the order of 0.4 cm, e.g., (0.4 cm)³.

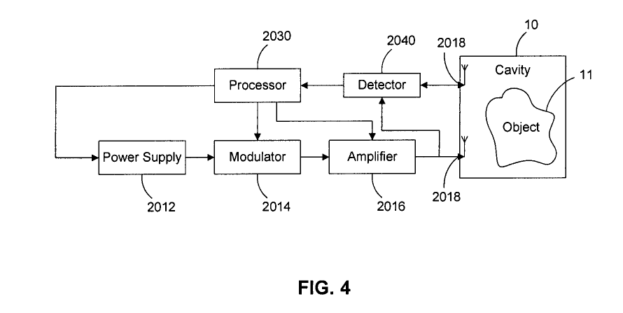

Reference in now made to FIG. 4, which provides a diagrammatic representation of an exemplary apparatus 100 for applying EM energy to an object, in accordance with some embodiments of the present invention. In accordance with some embodiments, apparatus 100 may include a processor 2030 which may regulate modulations performed by modulator 2014. In some embodiments, modulator 2014 may include at least one of a phase modulator, a frequency modulator, and an amplitude modulator configured to modify the phase, frequency, and amplitude of the AC waveform, respectively. Processor 2030 may alternatively or additionally regulate at least one of location, orientation, and configuration of each radiating element 2018, for example, using an electro-mechanical device. Such an electromechanical device may include a motor or other movable structure for rotating, pivoting, shifting, sliding or otherwise changing the orientation and/or location of one or more of radiating elements 2018. Alternatively or additionally, processor 2030 may be configured to regulate one or more field adjusting elements located in the energy application zone, in order to change the field pattern in the zone.

In some embodiments, apparatus 100 may involve the use of at least one source configured to deliver EM energy to the energy application zone. By way of example, and as illustrated in FIG. 4, the source may include one or more of a power supply 2012 configured to generate electromagnetic waves that carry EM energy. For example, power supply 2012 may be a magnetron configured to generate high power microwave waves at a predetermined wavelength or frequency. Alternatively, power supply 2012 may include a semiconductor oscillator, such as a voltage controlled oscillator, configured to generate AC waveforms (e.g., AC voltage or current) with a constant or varying frequency. AC waveforms may include sinusoidal waves, square waves, pulsed waves, triangular waves, or another type of waveforms with alternating polarities. Alternatively, a source of EM energy may include any other power supply, such as electromagnetic field generator, electromagnetic flux generator, or any mechanism for generating vibrating electrons.

In some embodiments, apparatus 100 may include a phase modulator (not illustrated) that may be controlled to perform a predetermined sequence of time delays on an AC waveform, such that the phase of the AC waveform is increased by a number of degrees (e.g., 10 degrees) for each of a series of time periods. In some embodiments, processor 2030 may dynamically and/or adaptively regulate modulation based on feedback from the energy application zone. For example, processor 2030 may be configured to receive an analog or digital feedback signal from detector 2040, indicating an amount of EM energy received from cavity 10, and processor 2030 may dynamically determine a time delay at the phase modulator for the next time period based on the received feedback signal.

In some embodiments, apparatus 100 may include a frequency modulator (not illustrated). The frequency modulator may include a semiconductor oscillator configured to generate an AC waveform oscillating at a predetermined frequency. The predetermined frequency may be in association with an input voltage, current, and/or other signal (e.g., analog or digital signals). For example, a voltage controlled oscillator may be configured to generate waveforms at frequencies proportional to the input voltage.

Processor 2030 may be configured to regulate an oscillator (not illustrated) to sequentially generate AC waveforms oscillating at various frequencies within one or more predetermined frequency bands. In some embodiments, a predetermined frequency band may include a working frequency band, and the processor may be configured to cause the transmission of energy at frequencies within a sub-portion of the working frequency band. A working frequency band may be a collection of frequencies selected because, in the aggregate, they achieve a desired goal, and there is diminished need to use other frequencies in the band if that sub-portion achieves the goal. Once a working frequency band (or subset or sub-portion thereof) is identified, the processor may sequentially apply power at each frequency in the working frequency band (or subset or sub-portion thereof). This sequential process may be referred to as “frequency sweeping.” In some embodiments, each frequency may be associated with a feeding scheme (e.g., a particular selection of MSEs). In some embodiments, based on the feedback signal provided by detector 2040, processor 2030 may be configured to select one or more frequencies from a frequency band, and regulate an oscillator to sequentially generate AC waveforms at these selected frequencies.

Alternatively or additionally, processor 2030 may be further configured to regulate amplifier 2016 to adjust amounts of energy applied via radiating elements 2018, based on the feedback signal. Consistent with some embodiments, detector 2040 may detect an amount of energy reflected from the energy application zone and/or energy applied at a particular frequency, and processor 2030 may be configured to cause the amount of energy applied at that frequency to be low when the reflected energy and/or coupled energy is low. Additionally or alternatively, processor 2030 may be configured to cause one or more antennas to deliver energy at a particular frequency over a short duration when the reflected energy is low at that frequency.

In some embodiments, the apparatus may include more than one EM energy generating component. For example, more than one oscillator may be used for generating AC waveforms of differing frequencies. The separately generated AC waveforms may be amplified by one or more amplifiers. Accordingly, at any given time, radiating elements 2018 may be caused to simultaneously transmit electromagnetic waves at, for example, two differing frequencies to cavity 10.

Processor 2030 may be configured to regulate the phase modulator in order to alter a phase difference between two electromagnetic waves supplied to the energy application zone. In some embodiments, the source of EM energy may be configured to supply EM energy in a plurality of phases, and the processor may be configured to cause the transmission of energy at a subset of the plurality of phases. By way of example, the phase modulator may include a phase shifter. The phase shifter may be configured to cause a time delay in the AC waveform in a controllable manner within cavity 10, delaying the phase of an AC waveform anywhere from between 0-360 degrees.

In some embodiments, a splitter (not illustrated) may be provided in apparatus 100 to split an AC signal, for example generated by an oscillator, into two AC signals (e.g., split signals). Processor 2030 may be configured to regulate the phase shifter to sequentially cause various time delays such that the phase difference between two split signals may vary over time. This sequential process may be referred to as “phase sweeping.” Similar to the frequency sweeping described above, phase sweeping may involve a working subset of phases selected to achieve a desired energy application goal.

The processor may be configured to regulate an amplitude modulator in order to alter amplitude of at least one electromagnetic wave supplied to the energy application zone. In some embodiments, the source of EM energy may be configured to supply EM energy in a plurality of amplitudes, and the processor may be configured to cause the transmission of energy at a subset of the plurality of amplitudes. In some embodiments, the apparatus may be configured to supply EM energy through a plurality of radiating elements, and the processor may be configured to supply energy with differing amplitudes simultaneously to at least two radiating elements.

Although FIG. 4 and FIGS. 2 and 3 illustrate apparatuses including two radiating elements (e.g., antennas 16, 18; 210, 220; or 2018), it should be noted that any number of radiating elements may be employed, and the circuit may select combinations of MSEs through selective use of radiating elements. By way of example only, in an apparatus having three radiating elements A, B, and C, amplitude modulation may be performed with radiating elements A and B, phase modulation may be performed with radiating elements B and C, and frequency modulation may be performed with radiating elements A and C. In some embodiments, amplitude may be held constant and field changes may be caused by switching between radiating elements and/or subsets of radiating elements. Further, radiating elements may include a device that causes their location or orientation to change, thereby causing field pattern changes. The combinations are virtually limitless, and the invention is not limited to any particular combination, but rather reflects the notion that field patterns may be altered by altering one or more MSEs.

Some or all of the forgoing functions and control schemes, as well as additional functions and control schemes, may be carried out, by way of example, using structures such as the EM energy application subsystems schematically depicted in FIG. 1 or FIG. 4. Within the scope of the invention, alternative structures might be used for accomplishing the functions described herein, as would be understood by a person of ordinary skill in the art, reading this disclosure.

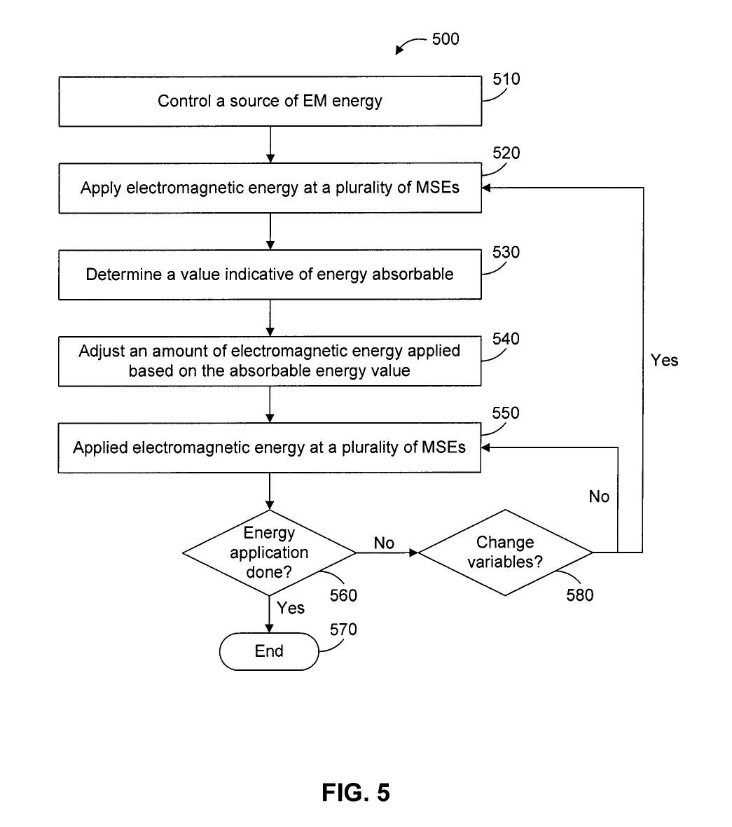

FIG. 5 represents a method for applying EM energy to an object in accordance with some embodiments of the present invention. EM energy may be applied to an object, for example, through at least one processor implementing a series of steps of method 500 of FIG. 5.

In certain embodiments, method 500 may involve controlling a source of EM energy (step 510). A “source” of EM energy may include any components that are suitable for generating EM energy. By way of example only, in step 510, the at least one processor may be configured to control EM energy application subsystem 96.

The source may be controlled to supply EM energy at a plurality of MSEs (e.g., at a plurality of frequencies and/or phases and/or amplitude etc.) to at least one radiating element, as indicated in step 520. Various examples of MSE supply, including sweeping, as discussed earlier, may be implemented in step 520. Alternatively or additionally, other schemes for controlling the source may be implemented so long as that scheme results in the supply of energy at a plurality of MSEs. The at least one processor may regulate subsystem 96 to supply energy at multiple MSEs to at least one transmitting radiating element (e.g., antenna 102). Additionally or alternatively, other schemes for controlling the source may be implemented. For example, one or more processing instructions and/or other information may be obtained from a machine readable element (e.g., barcode or RFID tag). The machine readable element may be read by a machine reader (e.g., a barcode reader, an RFID reader) and may be provided to the processor and/or the controller by an interface. In some embodiments, a user may provide one or more processing instructions and/or may provide other information relating to the object (e.g., an object type and/or weight) through an interface, e.g., a GUI interface, a touch screen etc.

In certain embodiments, the method may further involve determining a value indicative of energy absorbable by the object at each of the plurality of MSEs, in step 530. An absorbable energy value may include any indicator—whether calculated, measured, derived, estimated or predetermined—of an object's capacity to absorb energy. For example, computing subsystem 92 may be configured to determine an absorbable energy value, such as a dissipation ratio, the mean dissipation ratio and/or the input impedance associated with each MSE.

In certain embodiments, the method may also involve adjusting an amount of EM energy incident or applied at each of the plurality of MSEs based on the value indicative of energy absorbable at each MSE (step 540). For example, in step 540, at least one processor may determine an amount of energy to be applied at each MSE, as a function of the value indicative of energy absorbable associated with that MSE.

In some embodiments, a choice may be made not to use all possible MSEs. For example, a choice may be made not to use all possible frequencies in a working band, such that the emitted frequencies are limited to a sub band of frequencies, for example, where the Q factor in that sub band is smaller or higher than a threshold. Such a sub band may be, for example 50 MHz wide 100 MHz wide, 150 MHz wide, or even 200 MHz wide or more.

In some embodiments, the at least one processor may determine a weight, e.g., power level, used for applying the determined amount of energy at each MSE, as a function of the value indicative of energy absorbable. For example, amplification ratio of amplifier 2016 may be changed inversely with the energy absorption characteristic of object 11 at each MSE. In some embodiments, when the amplification ratio is changed inversely with the energy absorption characteristic, energy may be supplied for a constant amount of time at each MSE. Alternatively or additionally, the at least one processor may determine varying durations at which the energy is supplied at each MSE. For example, the duration and power may vary from one MSE to another, such that their product inversely correlates with the absorption characteristics of the object. In some embodiments, the controller may use the maximum available power at each MSE, which may vary between MSEs. This variation may be taken into account when determining the respective durations at which the energy is supplied at maximum power at each MSE. In some embodiments, the at least one processor and/or controller (e.g., controller 101) may determine both the power level and time duration for supplying the energy at each MSE.

In certain embodiments, the method may also involve applying EM energy at a plurality of MSEs (step 550). Respective weights are optionally assigned to each of the MSEs to be applied (step 540) for example based on the value indicative of energy absorbable (as discussed above). EM energy may be applied to cavity 10 via radiating elements, e.g., antenna 102, 16, 18 or 2018. In some embodiments, MSEs may be swept sequentially, e.g., across a range of cavity's resonance MSEs or, along a portion of the range.

Energy application may be interrupted periodically (e.g., several times a second) for a short time (e.g., only a few milliseconds or tens of milliseconds). Once energy application is interrupted, in step 560, it may be determined if the energy application should be terminated. Energy application termination criteria may vary depending on application. For example, for a heating application, termination criteria may be based on time, temperature, total energy absorbed, or any other indicator that the process at issue is compete. For example, heating may be terminated when the temperature of object 11 rises to a temperature threshold. If, in step 560, it is determined that energy application should be terminated (step 560: yes), energy application may end in step 570. In another example, in thawing application, termination criteria may be any indication that the entire object is thawed.

If the criterion or criteria for termination is not met (step 560: no), a determination may be made with regard to whether if variables should be changed and reset in step 580. If not (step 580: no), the process may return to step 550 to continue application of EM energy. Otherwise (step 580: yes), the process may return to step 520 to determine new variables. For example, after a time has lapsed, the object properties may have changed; which may or may not be related to the EM energy application. Such changes may include temperature change, translation of the object (e.g., if placed on a moving conveyor belt or on a rotating plate), change in shape (e.g., mixing, melting or deformation for any reason) or volume change (e.g., shrinkage or puffing) or water content change (e.g., drying), flow rate, change in phase of matter, chemical modification, etc. Therefore, it may be desirable to change the variables of energy application. The new variables that may be determined may include: a new set of MSEs, an amount of EM energy incident or applied at each of the plurality of MSEs, weight, e.g., power level, of the MSE(s) and duration at which the energy is applied at each MSE. Consistent with some of the presently disclosed embodiments, less MSEs may be swept in step 520 performed during the energy application phase than those swept in step 520 performed before the energy application phase, such that the energy application process is interrupted for a minimum amount of time.

In one respect, the invention may involve the use of EM/RF oven for processing object(s) by applying EM energy. The term ‘RF oven’ or ‘EM oven’ or ‘RF furnace’, ‘RF drying cabinets’, RF melting and smelting furnace', ‘RF remediation apparatus’ and ‘RF curing furnace’ as used herein, includes any device or apparatus that applies RF energy for cooking, heating, warming, making, preparing or any other processing on object(s), e.g., food object. An RF oven or an EM oven may comprise apparatus 100 described above in reference to FIG. 1 or FIG. 4 and may employ any suitable method such as those described above, for example method 500, for applying EM energy. RF oven or EM oven may be commercial or domestic oven.

Cooking Food in EM/RF Ovens

In some embodiments of the invention, object 11 may comprise at least one food product or food item, ready to be cooked, thawed, warmed, and/or browned. Object 11 may be a prepackaged food item or ready-to-eat meal or dinner that may comprise at least one food item. Food items in the prepackaged food or ready-to-eat meal may be cooked, heated or warmed up simultaneously in an energy application zone (e.g., energy application zone 9) optionally to reach a cooking condition or target for each of the various food items during the same cooking period. Optionally, the food item may be thawed prior to cooking or warming. Additionally or alternatively, the food item may be browned during or after cooking. Some examples of food items that may be treated in an EM energy application zone (e.g., zone 9) consistent with some embodiments of the present invention are listed below.

Different food items may have different ability to absorb EM energy based on their dielectric properties (e.g., dielectric losses, geometry and chemical composition). In some embodiments, a first and a second food item to be processed placed together in an energy application zone (e.g., energy application zone 9), may receive an amount of energy needed for reaching a requested or target cooking/thawing stage in parallel or simultaneously to one another. For example, a ready-to-eat dinner comprising: a beef steak, a broccoli and a potatoes salad, may be frozen at heating commencement, and a controller (e.g., controller 101) may control energy delivery or application to the energy application zone via a plurality of MSEs, for example according to the method described in FIG. 5, in order to first thaw all food items and then cook the steak to medium stage, cook the broccoli to al-dente stage and warm the potatoes salad to 10° C.

In some embodiments of the present invention, an energy application zone (e.g., energy application zone 9) may be a part of commercial or domestic oven. The oven may be instructed via an interface (e.g., interface 130) to cook the food item by applying EM energy using a plurality of MSEs, for example according to method 500 described in FIG. 5. Additionally or alternatively, the interface may instruct the oven to thaw the food item prior to cooking, and/or brown the food item after or during cooking to a desired level. In some embodiments, one or more processing on the object (e.g., browning) may be achieved by hot air impingement and not by applying RF energy. Optionally, the oven may be a dedicated oven, e.g., tailored to cook a specific food item(s) or product(s) for example: a pizza oven, or a pancake pan, or the like. The interface may receive instructions from one or more user interfaces, for example a GUI, or a barcode, RFID tag or the like (e.g., by using a reader, such as: RFID reader or barcode scanner), or from a remote location, e.g., an Internet server. The oven may include additional features such as hot air impingement unit(s), IR browning element(s), moisture control, temperature measurement element(s), deep oil frying pan, or the like. For example in an experiment done in a coking oven based on apparatus 100 and method 5 described in FIG. 1 and FIG. 5: yeast dough for preparing doughnuts was prepared in the usual manner, when the dough was ready for deep frying, it was backed in an RF oven until ready followed by deep frying for 5 seconds. The taste and texture was similar to deep fried dough, with optionally lower fat contents due to the very short actual frying process.

In some embodiments of the invention, the energy application zone may include at least one radiating element (e.g., antennas 102, 16, 18, 210 or 220) capable of emitting or applying EM energy at a plurality of MSEs, for example according to method 500 described in FIG. 5. Optionally, the energy application zone may include a pot, a casserole pot, a poyke, a pan or the like. The at last one radiating element may be located inside the energy application zone or outside the energy application zone, applying the EM energy via a window transparent to EM energy, or a combination of both.

A food product or an item processed in an energy application zone consistent with some embodiments, may include, for example, one or more of the following:

Pancakes, waffles, American waffles, blintzes, crêpes, Muffuletta or the like;

any type of yeast dough, either baked, fried or steamed for example: doughnuts, cinnamon or other rolls, Danish, Dim Sum or other dumplings or the like;

any type of bread or buns or rolls, either baked, fried and/or steamed including for example: croissant, baguette, brioche, Fougasse, flatbreads, Chapati, Phulka, Puri, Roti, Paratha, Naan, Kulcha, Bhatoora, Baqar Khani, Appam, Dosa, Luchi, Puran Poli, Pathiri, Porotta, Bring, Mantou, Focaccia, Grissini, Casatiello, Buccellato, Pane carasau, Panettone, Pita, Challah, matzoth, Pandesal, Pandeyuca, Cocol, mollete, Pan de coco, bagel, Rosca, tortilla, nachos, or the like;

any type of cake and cookies, for example: tourte, chocolate cakes, apple cakes, poppy-seed cakes, sponge cakes, orange cake, lemon cake, muffins, biscuits, or the like;

any kind of short pastry or puff pastry pie and tarte for example: apple pie, hazelnut pie, pecan pie, lemon pie, fruit pie, chocolate pie or the like;

any kind of cookie made from short pastry or puff pastry, either baked, fried or steamed for example: gingerbread, chocolate chips cookies, butter cookies or the like;

any kind of short pastry or puff pastry quiche, tête, caboche, crane, pâté or the like;

any stuffed dough either baked, fried or steamed for example: Pizza, calzone, Empanada, bourekas, Dim Sum, Won Tun, dumplings, pelmeni, piroski, or a dough stuffed with a whole chicken, roast beef, a whole turkey, or the like;

any dried or puffed cereals for example: popcorn, puffed rise, cornflakes, or any other cereal either made from a single ingredient or a mixture of several ingredients. The cereal may be coated with sweet, caramel, almonds, nuts, chocolate or other coating.

The food product may also include any kind of egg based mixture for example:

French toast, omelet, bread pudding, crème brûlée pudding, matzah brie, or the like;

any kind of vegetable or vegetable dishes, either baked, cooked, slow cooked, fried or steamed;

any kind of starchy vegetable either baked, cooked, slow cooked, fried or steamed, for example: potatoes, sweet potatoes, Jerusalem artichoke, pumpkin, squash, yam or the like;

any kind of grains or legume for example: Couscous, polenta, beans, chickpeas, Fava beans or the like;

any kind of rice based dishes (any type of white, brown, red, wild, or a mixture of two or more types of rice) for example: risotto, fried rice, pilaf, Risi e Bisi, paella, pulao, Domburi, Sushi, Onigiri, chazuka, kayu, curry, Bibimbap, Bokkeumbap, or the like;

any kind of meat for example: beef, veal, chicken, turkey, pork, game, deer, caribou, venison, rabbit, pheasant, moose, buffalo, duck, duckling, goose, mallard, or the like, baked, roast, cooked, slow cooked, steamed and/or fried;

any kind of fish baked, cooked, roast and/or steamed, for example: tuna, salmon, trout, halibut, swordfish, red mullet, cod, bass, sole, or the like;

any kind of seafood, baked, cooked roasted and/or steamed, for example: clams, crabs, mussels, lobster, langoustines, mollusks, shrimps, prawns, shellfish, octopus, calamari, or the like;

any kind of marmalade, jam, jelly, curd or confiture, for example: Dolce de Lecce, cherry tomatoes jam, onion jam, lemon curd or the like; and

any kind of soup, stew, stock or sauce, for example: chicken stock, brown veal stock, fish stock, court bouillon, tomato sauce, clam-chowder, minestrone, goulash or the like.

Curing Polymers

Curing polymers using RF energy at one or a plurality of MSEs may have benefits over conventional microwave methods for curing polymers. For example, uniform heating in desired areas, (e.g., predefined areas) may be obtained. Curing polymers that are transparent to conventional microwave radiation may be achieved by utilizing EM radiation at frequencies not usually used, additionally or alternatively to using a plurality of MSEs, thus eliminating the need to use dielectric fillers. Conventional and other microwave methods for curing polymers, may include adding dielectric fillers to polymers to allow their curing. Dielectric fillers are RF or microwave coupling materials, such as silicon carbide whisker (SiCw), metallic powder, graphite powder or others, capable of absorbing RF/EM energy.

In some embodiment of the invention, apparatus 100 may be utilized to cure polymers; and energy application zone 9 may be part of, or at least partially located in an RF curing furnace. Object 11 may be at least partially made of bulk material, for example, rubber, and/or epoxy reinforced with carbon-fibers or glass-fibers. The bulk material part or object may be shaped in a mold, using for example injection molding, extrusion or the like, to a final desired shape. In some embodiments, the at least one part may be placed inside the RF curing furnace for curing. A Controller (e.g., controller 101) may control at least one radiating element (e.g., antennas 102, 16, 18, 210 and 220) to apply low powered RF energy by sweeping over a plurality of MSEs in order to determine a value indicative of EM energy absorbable in the polymer (e.g., the dissipation information or dissipation ratio) of the at least one part and to choose MSEs and their respective weights needed in order to cure the polymer, for example according to method 500. Optionally, a protective atmosphere may be added to the RF curing furnace during the curing process. Curing RF furnace or oven according to some embodiments of the invention may be any apparatus configured to apply RF energy to cure a polymer(s). The RF curing furnace may include at least some of the components of apparatuses 100 illustrated in FIG. 1 and FIG. 4.

In some embodiments of the invention, object 11 may comprise one or more thin polymer layers that may be sprayed or molded on at least one semiconductor or any microelectronic device. The semiconductor or the microelectronic device may be inserted into the RF curing furnace. Low powered RF energy may be applied by, for example, sweeping over a plurality of MSEs in order to determine a value indicative of EM energy absorbable in the object (e.g., the dissipation information) of the at least one semiconductor or microelectronic device and to choose MSEs and their respective weights needed to cure the polymer layer. Then, high power RF energy may be applied at the chosen MSEs and their respective weights, for example according to method 500. Optionally, a protective atmosphere, for example N₂ or Ar may be added to the RF curing furnace during the curing process.

In some embodiments of the invention, object 11 may be at least one part manufactured in a rapid prototyping process using three dimensional (3D) model created by a CAD (computer aided design) program. The CAD program may create or output slices of the 3D model and the part(s) in accordance to the thickness of a polymeric layer that may construct the part(s). The part(s) may be manufactured by placing thin polymer layers, optionally epoxy, corresponding to the slices, one on top of the other. Optionally, different materials may be used for different layers (e.g., conductive and non-conductive layers) alternately. A first thin layer of liquid polymer may be created on a substrate according to the 3D model optionally by ionographic printing techniques or electrically activated spray nozzle. The substrate and the first layer may be placed in an RF curing furnace similar to the one described above, and low powered RF energy may be applied by sweeping over a plurality of MSEs in order to determine a value indicative of EM energy absorbed (e.g., the dissipation information) of the layer and to choose the MSEs and their respective weights needed to cure the polymer layer, for example according to method 500. Optionally, a protective atmosphere may be added to the RF curing furnace during the curing process. After curing the first layer, a second layer may be placed/sprayed/printed on top of the first and the process may repeat itself, until layers are placed and cured and a 3D device is created.

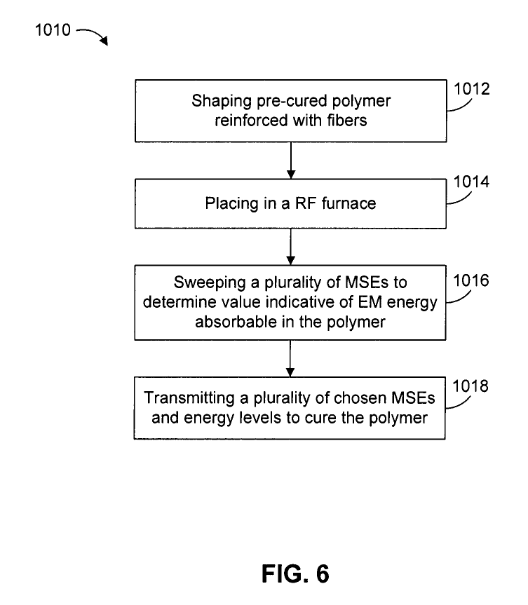

Referring now to FIG. 6 and flowchart 1010 illustrating a process for curing bulk parts made from polymer in accordance with some embodiments of the present invention. In some embodiments, at least one part made of pre-cured polymer reinforced by fibers may be shaped (step 1012), and placed in an RF curing furnace, step 1014. Low powered RF energy may be applied at a plurality of MSEs (e.g., swept through the plurality of MSEs) to determine a value indicative of EM energy absorbable by the polymer (e.g., dissipation information) at the part, step 1016. Based on the value determined in step 1016, at least one MSE and its respective weight may be chosen to be applied to cure the part in step 1018, for example according to method 500.

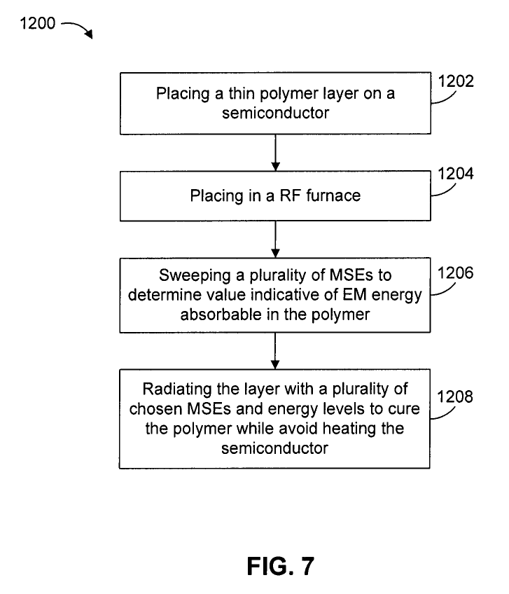

Referring now to Flowchart 1200 in FIG. 7, presenting an RF curing process (e.g., used in the microelectronic industry) in accordance with some embodiments of the present invention. A thin polymeric layer may be placed, sprayed, printed or otherwise applied on at least one a semiconductor wafer (step 1202), for example. Optionally, a thin polymeric layer may be applied to other microelectronic devices, for example to bond two microelectronic devices together, step 1202. The at least one microelectronic device or a wafer may be placed or inserted to an RF curing furnace (e.g., zone 9) in step 1204, and low power RF energy may be applied at a plurality of MSEs to determine value indicative of EM energy absorbable in the polymer and/or the semiconductor (e.g., dissipation information), in step 1206. Based on dissipation information determined in step 1206, at least one MSE and its respective weight may be chosen to be applied to cure the layer in step 1208, optionally while avoiding heating the wafer or microelectronic device to which the polymer layer was applied, for example according to method 500.

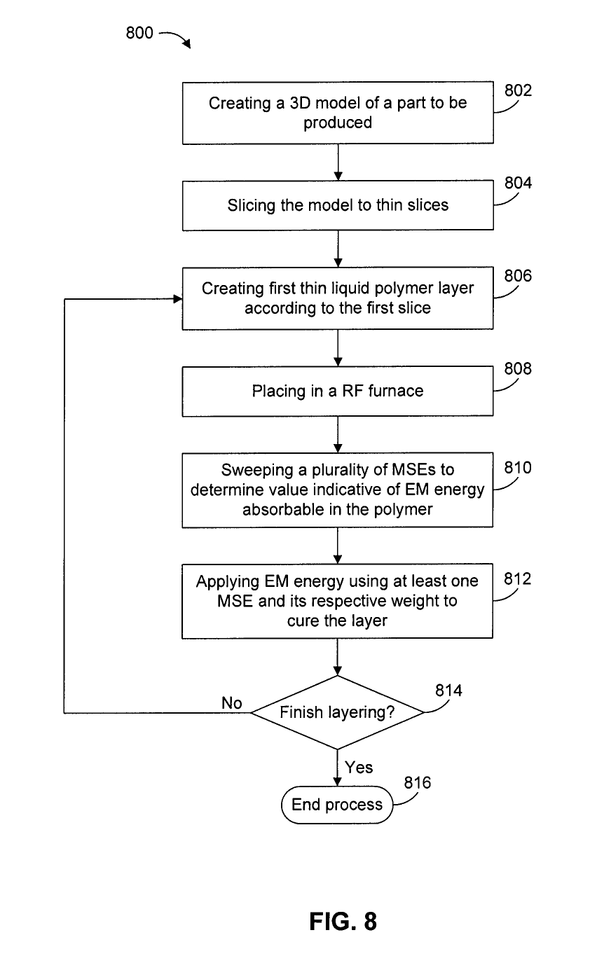

Reference is now made to FIG. 8, presenting a flowchart of a process 800 for rapid prototyping of three dimensional objects from thermo-set polymer, optionally epoxy, in accordance with some embodiments of the invention. A 3D model of the at least one part to be produced or manufactured may be created by a CAD software, step 802. For example a 3D model of the part to be manufactured can be designed using CATIA software, AutoCAD LT or any other CAD software. The CAD software may be used to simulate the behavior (e.g., mechanical strength) of the part based on known properties of the part's material(s). The CAD software may be additionally used to design the manufacturing process (CAM—computer aided manufacturing). In step 804, the software may “slice” the 3D model to various thin slices in accordance with the thickness of a polymeric layer designed to be applied. In step 806, a first thin polymer layer may be applied, on a substrate, according to the first slice from the 3D model, designed in step 804. For example, the polymeric layer may be applied by ionographic printing techniques or electrically activated spray nozzle. In step 808, the substrate may be placed in an RF curing furnace (e.g., energy application zone 9, FIG. 1), and low powered RF energy may be applied at a plurality of MSEs, e.g., by sweeping, to determine a value indicative of EM energy absorbable in the polymer layer (e.g., dissipation information), in step 810. Based on the dissipation information determined in step 810, EM energy (e.g., RF energy) at least one MSE and its respective weight may be chosen to be applied to cure the layer in step 812, for example according to method 500 described in FIG. 5. In some embodiments, a query is made to determine whether all layers are cured (step 814). If not all layers are cured (step 814: NO), a second layer may be produced, on the first layer, according to the second slice from the 3D model and steps 806-812 may be repeated until the questions, in step 814, whether all layers are cured is answered with a “yes” and the process in ended in step 816.

Sintering Processes

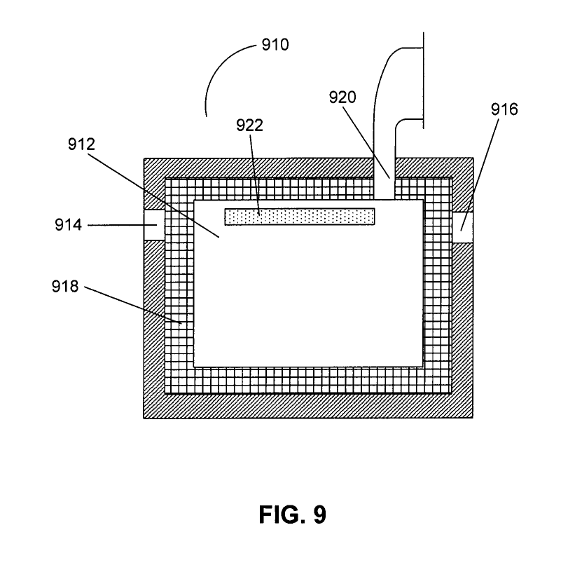

In some embodiments of the present invention, apparatus 100 may be used for sintering and/or processing parts, and energy application zone 9 may be located in an RF sintering or processing furnace, having a cavity for heating and/or treating objects for example, to be sintered. Optionally, the RF sintering or processing furnace may be partitioned by at least one shelf, in order to increase the capacity of the furnace. RF sintering or processing furnace according to some embodiments of the invention may be any apparatus configured to apply RF energy to sinter or process an object (e.g., a pressed green body). The embodiments disclosed below refer to a sintering furnaces, are given in a way of example only, and the apparatus disclosed in FIGS. 9-11 may be used to processed objects other than pressed powders to be sintered, for example: metal parts to be heat treated, microelectronic devices to be processed and/or annealed, ceramic and glass parts to be annealed, etc.

In some embodiments of the invention, at least one object to be sintered (e.g., exemplary object 11) may be placed in the RF sintering furnace. The object may be made from a pre-sintered and pressed powder. The powder may be, for example, made of metal, metal oxide, metal carbide, or a combination of two or more thereof. The object may be, for example, a “green body”, as this term is used in the art. Optionally, protective atmosphere may be applied to a cavity in the RF sintering furnace during heating. Optionally, additional convection heating may be applied to elevate the temperature of the object, e.g., in order to increase the absorption ability of the green body or one of its constituents, for example, a metal oxide. RF energy may be applied to the energy application zone (e.g., energy application 9, FIG. 1) at a plurality of MSEs in order to heat and sinter the object, optionally to increase the density of the sintered object. Additionally or alternatively, high pressure may be added during or after the heating process to increase the density of the sintered body by eliminating microporosity.

For example, the at least one object may be a green body made from metallic powder. The at least one object may be placed in the energy application zone. Optionally, a protective atmosphere may be applied to the RF sintering furnace. RF energy may be applied to the cavity at a plurality of MSEs in order to heat and sinter the object, optionally to increase the density of the sintered object, in some examples while avoiding melting the metallic power.

In some embodiments, the object to be sintered may be MMC (Metal Matrix Composite), made from a mixture of metallic and ceramic powders, for example cobalt and tungsten-carbide also known as cemented-carbide. The green body(s) may be placed in the energy application zone (e.g., to in a cavity), optionally a protective atmosphere may be applied, and RF energy may be applied to the cavity at a plurality of MSEs, in accordance to, for example, method 500, in order to sinter the objects, optionally while melting the metallic powder.

Referring to FIG. 9 illustrating an RF sintering furnace 910 in accordance with some embodiments of the present invention, having an energy application zone (e.g., cavity) 912 with two RF radiating elements 914 and 916. Energy application zone 912 may include a metallic cavity, comprising of at least one metallic wall. In some embodiment, the metallic walls may include cast iron, steel, cupper alloys etc. The furnace may further include power unit, e.g., source, (not shown) which may supply RF) energy to the radiating elements and a control unit, e.g., controller, (not illustrated) configured to control (e.g., adjust) energy application to energy application zone 912 in order to apply the RF energy required to sinter the object. Radiating elements 914 and 916 may be entirely located or partially located in energy application zone 912 or located outside energy application zone 912. When radiating elements 914 and 916 are located outside energy application zone 912, furnace 910 may contain two windows transparent to RF energy that may deliver the RF energy to energy application zone 912 (not illustrated). Energy application zone 912 may also include a refractory coating 918 made from RF transparent material. Optionally, furnace 910 may include a system 920 for applying Ar, N₂, CO₂, vacuum, high pressure, or other controlled atmosphere. Optionally, the furnace may include a convection heating element 922 to apply additional heat to energy application zone 912, for example by IR radiation or hot air impingement.

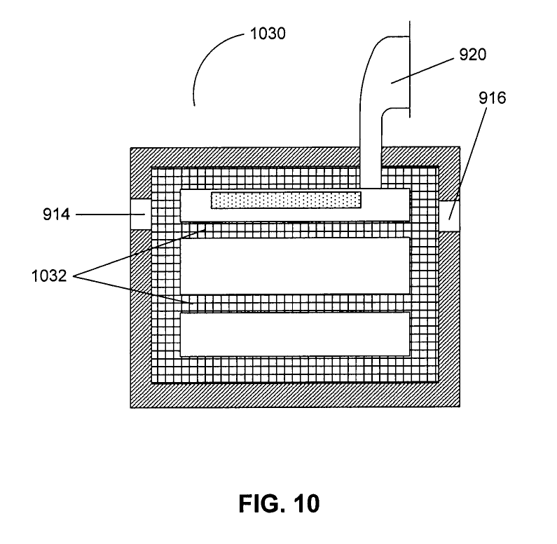

FIG. 10 illustrates an RF sintering furnace 1030 having similar components as furnace 910 illustrated in FIG. 9, and may further include a plurality of shelves 1032 made from a refractory RF transparent material and placed at desired heights. Optionally, the refractory RF transparent material may be electrical isolators for example metallic oxides such as Alumina, Silica, Zirconia, or a mixture of two or more metallic oxides.

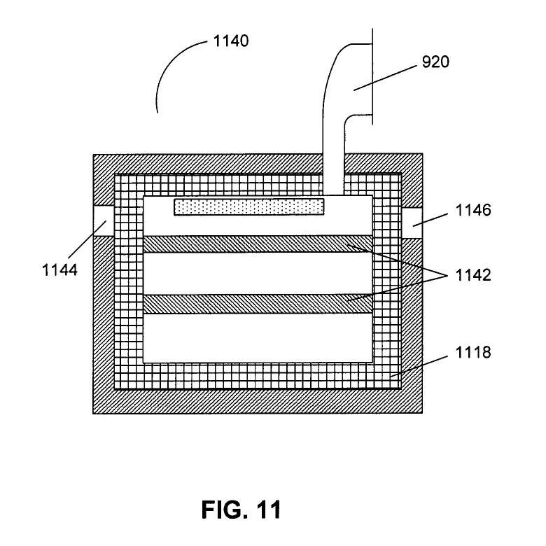

Reference is now made to FIG. 11 illustrating an RF sintering furnace 1140 having similar components as furnace 910 illustrated in FIG. 9, and may further have a partitioned energy application zone sintering furnace 1140 FIG. The energy application zone may further include metallic shelves 1142 isolated from the metallic cavity (e.g., zone 912) by a refractory coating 1118 similar to coating 918. RF sintering furnace 1140 may include two radiating elements 1144 and 1146; a power unit (not illustrated) which may generate electromagnetic (EM) energy to be supplied to the radiating elements and a control unit (not illustrated)—all configure to control the energy application to the energy application zone.

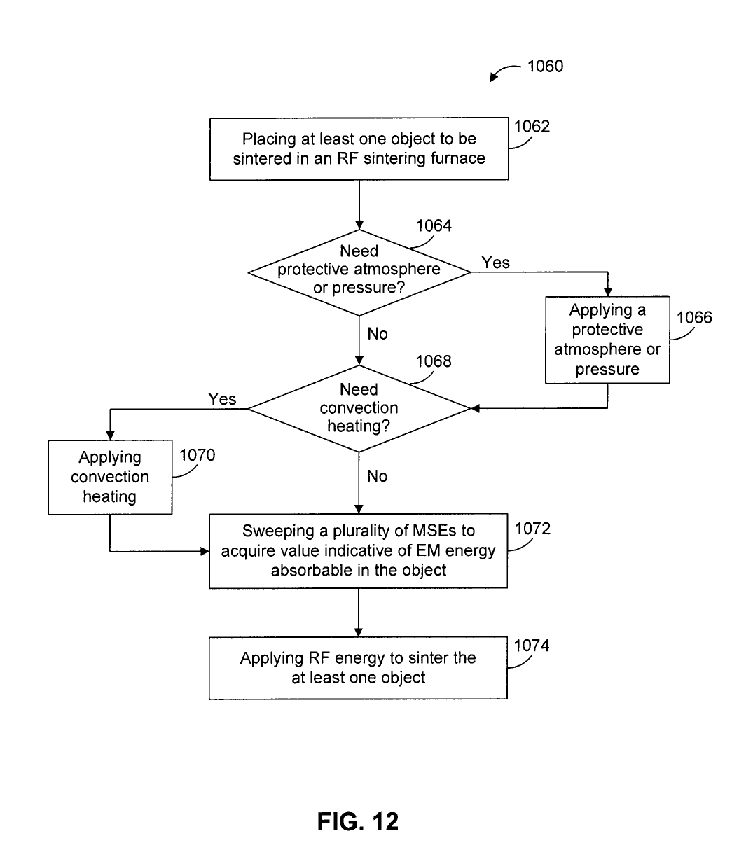

FIG. 12 illustrates a flowchart of method 1060 for sintering at least one object by utilizing RF energy according to some embodiments of the present invention. In step 1062, an object to be sintered may be placed in an energy application zone (e.g., zone 9 or 910) in an RF sintering furnace (e.g., furnaces 910, 1030 or 1140). In step 1064, it may be considered whether to apply protective atmosphere or pressure; and upon a positive decision (step 1064: YES) energy application zone may be either vacuumed or filled with the required gas in step 1066, by using for example system 920. Optionally, additional convection heating may be considered in step 1068 and may be applied if needed (step 1068: YES) in step 1070, by for example convection heating element 922. In step 1072, RF energy may be applied to the energy application zone and swept over a plurality of MSEs to acquire a value indicative of EM energy absorbable in the object. In step 1074, RF energy may be applied to the energy application zone to sinter the object via a plurality of MSEs based on the acquired value, for example according to method 500.

Drying Processes

Industrial use that requires extraction of moisture and liquids (both water-based and non-water-based) out of an object may utilize microwave or RF energy. Industrial and laboratory drying cabinets (i.e., RF drying cabinet) operated by a microwave may have better efficiency in terms of energy consumption than convection heating cabinets. The microwave or RF energy may penetrate the object (part to be dried) and may heat up the water or other liquid molecules in the part, thus most of the energy may be applied to the part to be dried and not the surrounding area.

In some embodiments of the invention, apparatus 100 (FIG. 1) may be an RF drying cabinet and energy application zone 9 may be at least partially located in the cabinet. RF drying cabinet according to some embodiments of the invention may be defined as an apparatus for processing an object using RF energy in order to reduce the amount of moisture and liquid in the object. Object 11 may be, for example: food items (e.g., fruits, nuts, cereals, etc) timber or wood product, a fabric, lost foam cluster and sand core, latex foams, X-Ray film, resins, trim base panels, glass fibers optionally on forming tubes, latex foams, ceramics, herbs leaves and flowers, fruits, grains, cereals and vegetables, and drying paint on various shaped parts. Optionally, residual moisture may be monitored during the drying process. Optionally, vacuum or other protective atmospheres may be used during the drying process to protect drying part and/or assist the drying process.

In some embodiment of the invention, energy application zone 9 may be near or at a printer and object 11 may be a drying ink. The drying may be carried out by applying EM energy at a plurality of MSEs to an element that radiates IR energy and thus the thin ink layer may be dried. Alternatively, EM energy may be applied directly to heat and dry the ink layer. For example, drying water based inks may benefit from utilizing MSEs that include frequencies in a range of 300-3000 MHZ that are better absorbed by water. Other inks may benefit the use of other RF frequencies or frequency ranges.

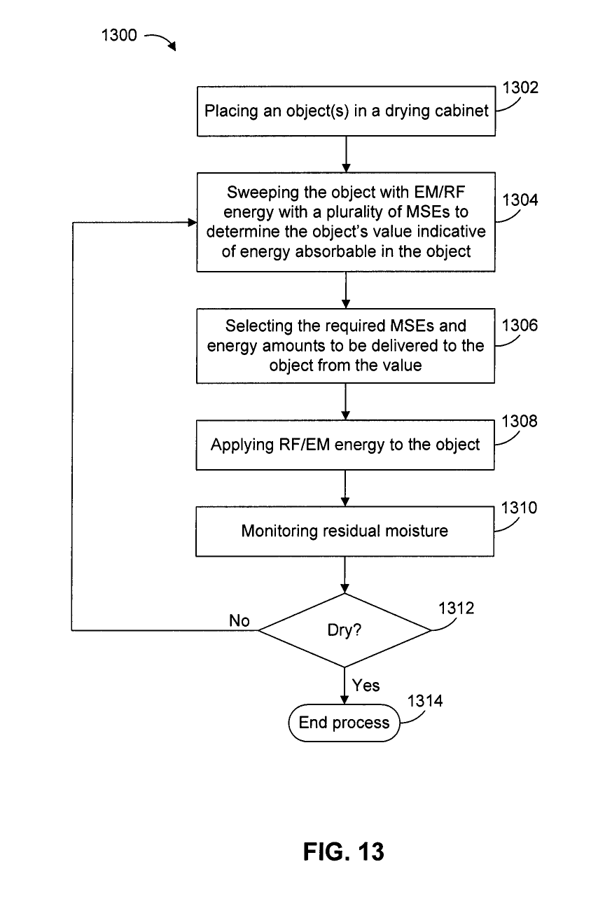

Referring now to FIG. 13 illustrating method 1300 for drying objects by applying RF energy in accordance with some embodiments of the invention. In step 1302, an object to be dried may be placed in an RF drying cabinet. The object may contain a single part or a plurality of parts to be dried. Optionally, only a portion of the object placed in the RF drying cabinet may require drying, for example, only the paint of a car door may be dried. RF energy may be initially applied to the object to sweep a plurality of MSEs to acquire value indicative of EM energy absorbable in the object (e.g., dissipation information) (step 1304). Based on the value acquired in step 1304, RF energy may be applied at one or more MSEs (e.g., by assigning corresponding weights (e.g., amount of energy) to each MSE) in step 1306 and applied to the object in step 1308. The method described herein may be in accordance with and corresponding to the method described in FIG. 5. Optionally, the residual moisture of the object may be monitored (e.g., by a hygrometer) to maintain a desired level (step 1310). In step 1312; a query is made to determine whether the object is dry. As long as the object is not dry (step 1312: NO (e.g., as long as the monitored moisture is at high level) steps 1310-1312 may be repeated. When the monitored moisture decreases to below a predetermined level (step 1312: YES), the process may end at step 1314.

Smelting and Melting of Metals and Ores

Bulk metals are known to be very good EM reflectors especially in the RF range, however, powders or particles of metals, metallic oxides and ores are good RF energy absorbers. Metallic powders may heat up by utilizing (applying) RF energy, due for example high electric currents on the surface of each powder particle. In the same manner, metallic oxides and ores may be dielectrically heated utilizing RF energy. Some ores behave like dielectric material while other behave like semi-metals. A chemical reduction agent may be added to change the oxidation state of the metal ore. The reducing agent is usually a carbon or carbon monoxide that removes oxygen from the ore to leave the metal.

In some embodiments of the invention, apparatus 100 may be an RF smelting furnace. RF smelting furnace according to some embodiments of the invention may be any apparatus configured to apply RF energy to process an object in order to smelt the object. Energy application zone 9 may be at least partially located in the RF smelting furnace, and object 11 may be metallic ores. An RF heating process may be aimed at producing molten metal from the metallic ores. The smelting furnace may take the form of a tall chimney-like structure, lined with refractory bricks. The chimney may have an opening at the top for receiving continuous supply of ores. The same or other opening may be used for receiving the chemical agent and flux. A flux may be, for example, a mineral added to the metals in a furnace to promote fusing or to prevent the formation of oxides. The smelting furnace may further include two bottom openings, a first for removal of the slag, and a second for collecting the molten metal. The second opening may be lower than the first.

The chimney may comprise at least one radiating element. The radiating element may be placed at the chimney's middle section, also known as the reaction zone. The radiating element may be placed inside the reaction zone and may be covered by a protective, refractory cover, or placed outside a chimney having RF transparent window, or a combination of both. The at least one radiating element may be connected to a power unit which may supply RF energy to the radiating element(s) at a plurality of MSEs, and to a control unit (e.g., controller 101). The control unit may be configured to adjust the application of RF energy at the plurality of MSEs in accordance with a value indicative of EM energy absorbable in the metal powders or ores (e.g., dissipation information), according to, for example, method 500 disclosed in FIG. 5. Optionally, hot air blasting and/or oxygen gas may be added to the smelting furnace to accelerate the chemical reduction of the ores.

In some embodiments of the invention, apparatus 100 may be a melting RF furnace. RF melting furnace according to some embodiments of the invention may be any apparatus configured to apply RF energy to process an object in order to melt the object. Energy application zone 9 may be at least partially located in the melting RF furnace and object 11 may be metal powder, or grained metal, or a combination of metal powder with bulk metal. The object may be introduced to a crucible coated with refractory coating, located inside the furnace. At least one radiating element may be placed inside the crucible covered by a protective refractory cover. Alternatively or additionally, the radiating element(s) may be placed outside the crucible, optionally including an RF transparent window. The at least one radiating element may be connected to a power unit which may supply RF energy to the radiating element(s) at a plurality of MSEs, and a control unit may be configured to adjust the energy application, according to for example the method described in FIG. 5. Optionally, protective atmosphere, for example, Vacuum, Ar, CO₂ or N₂ may be applied during melting. Additional fluxes aimed to further clean the metal and/or additional alloying elements may be added during the furnace operation to produce a metallic alloy. The furnace may include a tilting device to tilt the furnace and extract the molten metal/alloy.

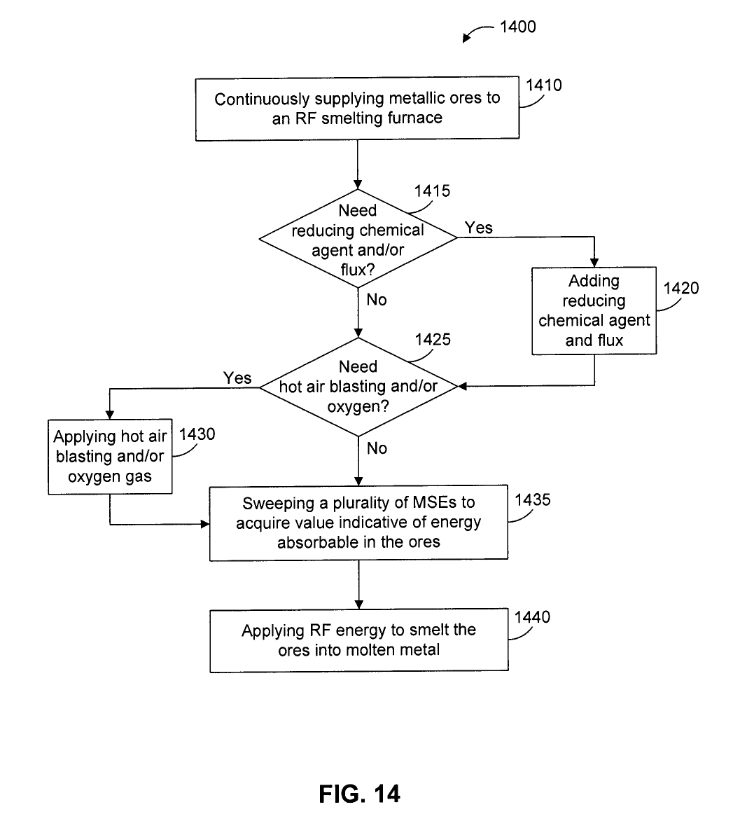

Referring now to flowchart 1400 and FIG. 14, metallic ore may be continuously supplied to an RF smelting furnace (step 1410). Optionally reducing chemical agent (minerals aimed to clean the molten metal from oxides) and/or flux may be considered in step 1415 and added if required (step 1415: YES) in step 1420. Optionally, hot air blasting and/or oxygen may be considered in step 1425 and added if required (step 1430: YES). RF energy may be initially applied to the object to sweep a plurality of MSEs to acquire a value indicative of EM energy absorbable in the metallic ores (e.g., dissipation information) and the additives, in step 1435. RF energy may be applied in order to smelt the ores into molten metal, in step 1440, for example according to method 500 described in FIG. 5. For example, RF energy application may be adjusted based on the value indicative of EM energy absorbable in the metallic ores.

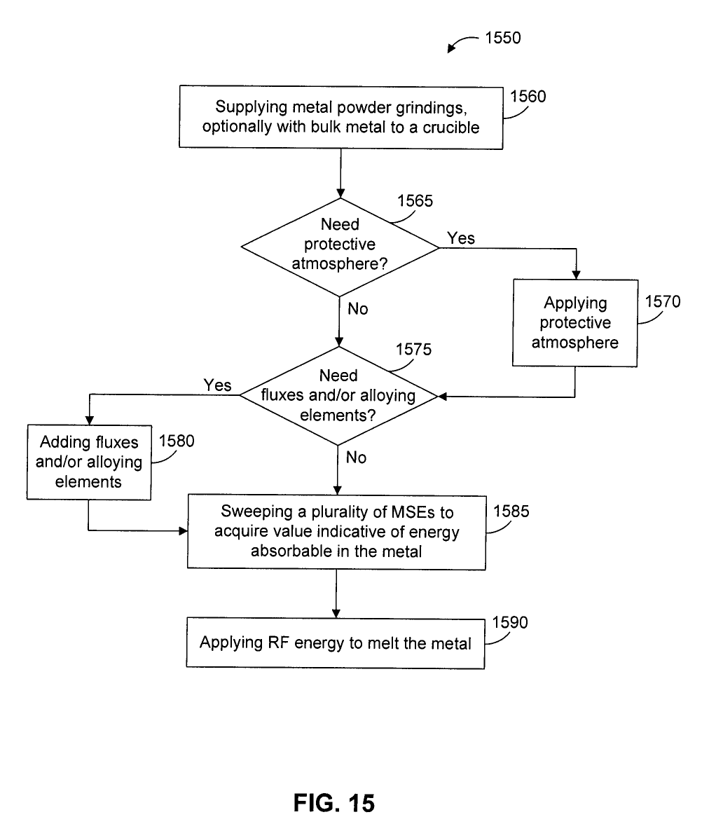

Referring now to flowchart 1550 and FIG. 15, metal in a form of metal powders, or grained metals, or a combination of metal powder with bulk metal may be supplied to a crucible in a melting furnace (step 1560). In some embodiments, protective atmosphere may be considered in step 1565 and may be applied if required (step 1565: YES) in step 1570. In some embodiments, fluxes and/or alloying elements may be considered in step 1575 and may be added if required (step 1575: YES) in step 1580. EM energy may be initially applied to the metal to sweep a plurality of MSEs in order to acquire a value indicative of EM energy absorbable in the metallic powder (e.g., dissipation information) and the additives, in step 1585. RF energy may be applied in accordance with the acquired value indicative of EM energy absorbable in the metallic powder to melt the metal into molten metal, in step 1590 (for example according to method 500 described in FIG. 5).

Soil Remediation

Microwave and RF energy remediation or reclamation has become a method to treat soils, sediments, and sludge. Hazardous compound such as PCB (polychlorinated biphenyls), carbon tetrachloride carbon tetrachloride, 1,1,1-trichloroethane and HCB (hexachlorobenzene) are some known common soil contaminators; all may be treated successfully with Microwave and RF energy. Microwave radiation may penetrate the soil and may heat water and contaminants within the soil. Vapors of the heated water (or other liquids) may be developed and evaporated due to the application of RF energy to the soil and may be withdrawn from the soil. The process may be rapid as compared to other methods, and its efficiency may depend on the dielectric and physicochemical properties of the soil and the contaminant. The process may allow the removal of volatile and semi-volatile components, and may be especially effective in the case of polar compounds.

In some embodiments of the invention, object 11 may be a contaminated soil to be remediated and apparatus 100 may be an RF batch remediation applicator optionally having a closed cavity. Energy application zone 9 may be at least partially located in the RF remediation applicator. An RF remediation apparatus according to some embodiments of the invention may be any apparatus configured to apply RF energy to process an object in order to clean the object from hazardous compound. The embodiments disclosed below refer to the remediation of soil, are given as examples only, thus the invention is not limited to soil remediation. For example the invention may be used to clean contaminated water, to recycle waste, etc. At least one radiating element may be located inside the cavity for applying RF energy to the contaminated soil. Optionally, the at least one radiating element may be at least partially inside the soil. Additionally or alternatively, the radiating element(s) may be a leaky wave antenna. The RF remediation applicator may also include air flow system for pumping out and collecting the evaporated hazardous gasses and may include one or more thermocouple or other sensor for monitoring the soil's temperature. The remediation applicator may further include additional sensors) (e.g., for monitoring the soil's humidity and residual organic contamination).

In some embodiments, apparatus 100 may be an open bench scale applicator. Contaminated soil may be continually added to a conveyor (e.g., a conveyor belt) to enter an open cavity. In the cavity, RF energy may be applied to the conveyed soil at a plurality of MSEs using at least one radiating element. During conveying in the cavity the contamination, e.g., organic contamination, may be heated up and evaporated until a desired level of cleanness is achieved in the exit end of the cavity. Optionally, several radiating elements or a long leaky wave antenna may be located along the cavity from one end to the other. Optionally, the radiating elements may be located under the conveyor. The applicator may also include air flow system for pumping out and collecting the evaporated hazardous gasses and optionally at least one thermocouple (or other sensor) for monitoring the soil's temperature. It may further include additional sensors for monitoring the soil's humidity and residual organic contamination.

In some exemplary embodiments of the invention, the soil may be contaminated with PCB (polychlorinated biphenyls), carbon tetrachloride, 1,1,1-trichloroethane and HCB (hexachlorobenzene) or the like. Irradiation of the contaminated soil may result in a remediation of the soil to a required level.

In some embodiments of the invention, powder particles, for example Fe, MnO₂, graphite, carbon fibers or other particulate matter may be added to the contaminated soil as strong RF absorbers prior to delivering or placing the soil in the applicator. Optionally, polar organic solvents may be added to the contaminated soil for both improving RF/EM absorption and affecting the breakdown or destruction of the organic contaminates into simpler, safer products.

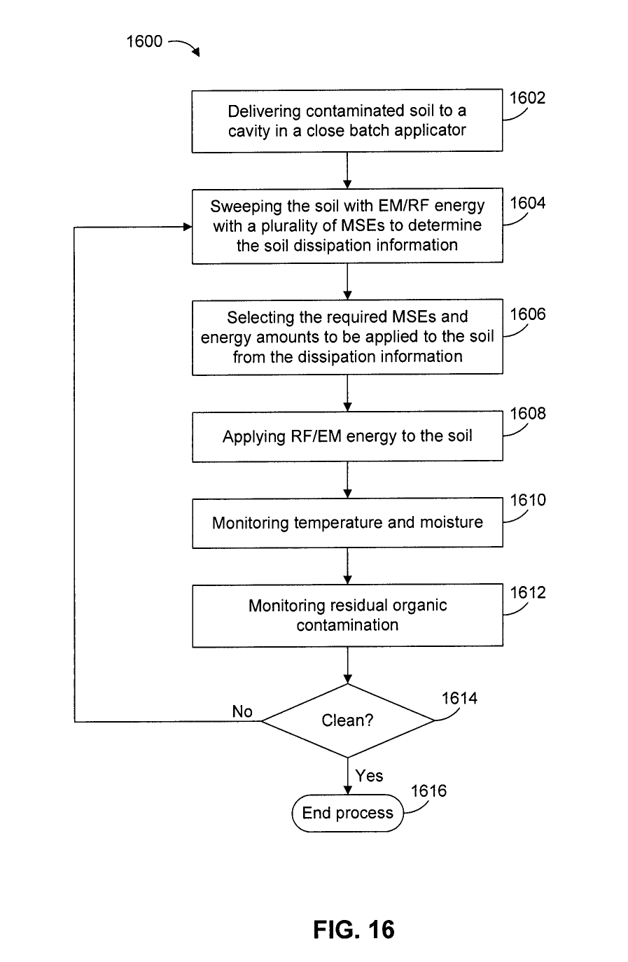

Referring now to FIG. 16 and flowchart 1600, in step 1602 a contaminated soil may be applied into a closed batch applicator. RF energy may be initially applied to the soil by sweeping a plurality of MSEs in order to determine the soil's value indicative of EM energy absorbable at each of the MSEs (step 1604). Based on the value indicative of EM energy absorbable determined in step 1604, at least one MSE and corresponding weight (e.g., energy amount) may be selected in step 1606 to be applied to at least a portion of the soil in step 1608, according to for example method 500 described in FIG. 5. Optionally, the temperature and/or the moisture of the soil may be monitored to maintain a desired level in step 1610 and the application of RF energy may be adjusted according to the monitored temperature and/or the moisture of the soil. Optionally, residual contamination levels may be monitored in step 1612 for determining whether the soil is cleaned and remediated, in step 1614. If not (step 1614: NO) steps 1610-1614 may be repeated, else (step 1614: YES) the process is ended at step 1616.

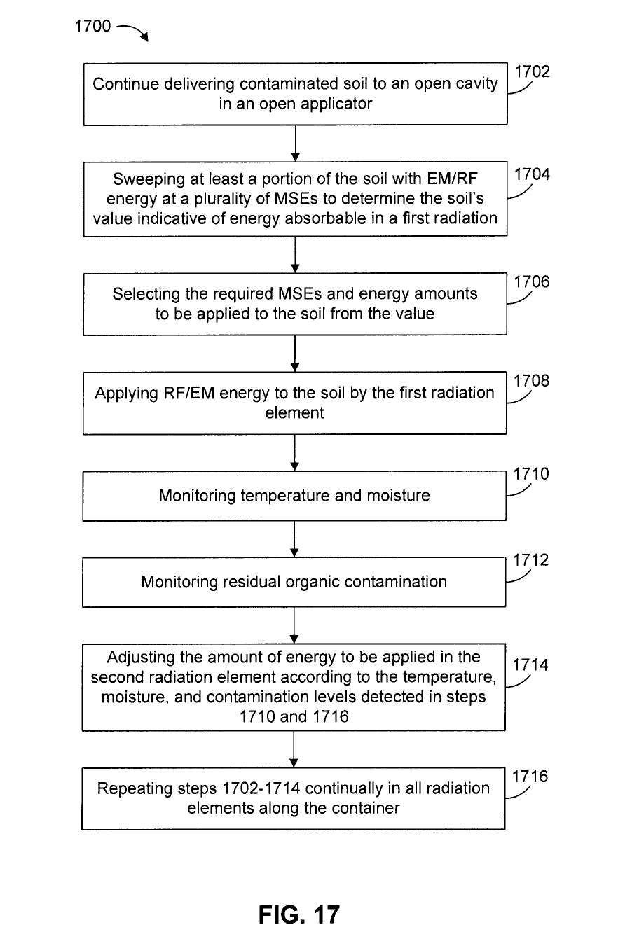

Reference is now made to FIG. 17 and flowchart 1700, illustrating a continuous process for remediation of contaminated soil in an open bench RF applicator in accordance with some embodiments of the invention. Contaminated soil may be continuously delivered to the entrance end of an open RF applicator optionally comprising a plurality of radiating elements along its path (step 1702) or a single slotted waveguide. A slotted waveguide according to some embodiments may be a longitudinal waveguide comprising at least one source of RF energy (e.g., a radiating element) at one end of the waveguide and have two or more slots along the waveguide for emitting the RF energy to an energy application zone (e.g., a soil remediated). Low RF energy may be initially applied to the soil, by a first radiating element or a first array of radiating elements, by sweeping over a plurality of MSEs in order to determine a value indicative of EM energy absorbable in the soil at each of the plurality of MSEs (step 1704). Based on the determined value in step 1704, at one or more MSEs and corresponding weights (e.g., energy amounts) may be selected in step 1706 and may be applied to at least a portion of the soil in step 1708, for example according to method 500. Optionally, the temperature and/or the moisture level of the soil may be monitored at the soil portions that were treated by RF energy, in step 1710. Optionally, residual contamination levels at the same soil portions may be monitored in step 1712. Optionally, information gathered in step 1710 and/or 1712 may be sent to the controller (e.g., controller 101) (step 1714), and the amount of EM/RF energy to be applied at a second radiating element or a second array of radiating elements may be adjusted based on the gathered information. Additionally or alternatively, the amounts of RF energy to be applied at a second radiation element may be adjusted based on the information gathered from the soil after passing through the first radiating element and dissipation information determined for the second radiation element in step 1704. The above process may be repeated for all radiating elements along the applicator (step 1716).

RF Plasma

Artificial plasmas can be divided into two major groups: thermal plasma and non-thermal or “cold” plasma. This grouping is based on the relationships between three temperatures: the excitation temperature of the plasma (Tₑ), the temperature of the ionized atoms in the plasma (Tᵢₒₙₛ) and the temperature of the gas (T_{gas}). Plasma is considered thermal if Tₑ=Tᵢₒₙ=T_{gas}, and non-thermal or “cold” plasma if Tₑ>>Tᵢₒₙ≈T_{gas}.

Non thermal plasma is generated by the application of DC or RF electric field to the gap between two metal electrodes, or electrode consists of a coil wrapped around a discharge volume. The applied RF energy may be low frequency RF (e.g., having a frequency of less than 100 kHz) and/or high frequency RF, e.g., having a frequency above 13.56 MHz. Non-thermal plasma may be, for example, low-pressure plasma (below atmospheric pressure) or atmospheric pressure plasma.

High frequency RF plasma may be used in the micro-fabrication and integrated circuit manufacturing industries for plasma etching and plasma enhanced chemical vapor deposition and crystal growth. High frequency RF plasma can also be applied for decomposition of VOC's (volatile organic compounds).

Thermal plasma is usually generated at atmospheric pressure by high power thermal discharge of very high temperature (2,000-10,000K). It can be generated using high powered microwaves or RF sources. This electrode-less plasma generator may be used to generate plasma torches for metal cutting or welding applications and waste disposal by thermal plasma pyrolysis. Plasma pyrolysis can be described as the process of reacting a carbonaceous solid with limited amounts of oxygen at high temperature to produce gas and solid products.

In some embodiments of the invention, object 11 may be a gas to be ionized, for example Ar, N₂, O₂, He, CH₄, or the like, and may be further inserted to an energy application zone (e.g., zone 9) optionally having two metal electrodes or a coil wrapped around the discharge volume. EM energy may be applied at a plurality of MSEs to the energy application zone from at least one radiating element for charging the electrodes, for example according to method 500. Non thermal plasma may be generated in accordance with some embodiments by electrically ionizing of the gas in either low pressure or atmospheric pressure. The ionized gas may be used for plasma etching or plasma enhanced chemical vapor deposition and crystal growth, or micro fabrication or any other method utilizing non-thermal plasma.

In some embodiments for processing thermal plasma, the gas may be irradiated by high intensity EM energy using a plurality of MSEs, for example according to method 500, to elevate the temperature of the gas to temperature higher than 2,000K. The high temperature may thermally discharge the gas to form high temperature plasma that may be used to generate plasma torches for metal cutting or welding applications or waste disposal or any other method utilizing thermal plasma.

In some embodiments, organic and non-organic waste may be placed in a plasma reactor for thermal plasma pyrolysis.

Pet Food

In some embodiments the object may be pet food to be cooked and the energy application zone may be at least partially located inside a pet food cooking apparatus. In general, pet food ingredients includes one or more of meat, meat byproducts, poultry, seafood, cereals, grain, liquid ingredients, vitamins, and minerals. Pet food may include dry food, canned food and semi-moist food. In some cases, animal parts used for pet food may include bones, bones flour, and cheek meat, and internal organs such as intestines, kidneys, liver etc or other parts, such as: feathers or mill. Cereal grains, such as: soybean meal, cornmeal, cracked wheat or barley, are often used to improve the consistency of the product. Liquid ingredients may include water, meat broth, or blood. Additionally, salt, preservatives, stabilizers, and gelling agents may be used. In some cases, artificial flavors may be used as well. It should be noted that the pet food may be vegetarian food (e.g., for non-carnivorous animals) for example birds.

Conventional methods for making pet food may include one or more of the following steps:
(1) Rendering flesh products to separate water, fat, and protein components.
(2) Grinding meat products to a desired texture and then cooking the meat mixture.
(3) Blending the meat mixture with other ingredients) (e.g., cereal grains, vitamins, and minerals).
(4) Heating the mixture. In some cases, to achieve the marbled-look of real meat, parts of the mixture may be cooked unevenly.
(5) Optionally shaping the mixture to the appropriate shape. Optionally, the steps of heating (4) and shaping (6) of the mixture may be combined, for example, in an extrusion process, for example: by the use of extruders.
(6) For canned food, a sterilization step may be performed, e.g., the cans are heated to about 121° C. and then are quickly cooled to about 38° C.

In accordance with some embodiments, a method for cooking and/or preparing pet food by applying RF energy may be faster and more efficient (in terms of energy consumption) compared to conventional methods for cooking pet food.

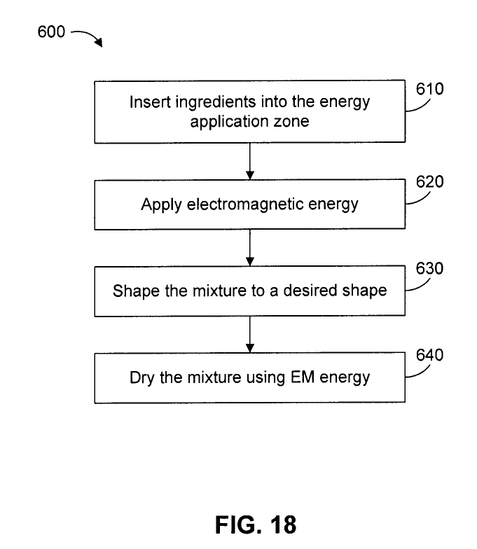

Reference is now made to FIG. 18 illustrating a method 600 for cooking and/or preparing a pet food by applying RF energy in accordance with some embodiments of the present invention.

In accordance with some embodiments, pet food ingredients may be inserted into the energy application zone (step 610). The ingredients may include: meat, a meat mixture, a meat mixture blended with other ingredients (e.g., cereal grains). At times, different ingredients may be added at different times during preparation. For example, a meat mixture may be heated first and after it was heated to a predefined temperature, other ingredients may be added for further heating. The energy application zone may include, for example, cavity 10 as illustrated, for example, in FIG. 2 or FIG. 4. The pet food may be cooked in an RF oven. The term RF oven, as used herein, includes any device that applies and/or supplies RF energy for cooking and/or heating and/or making and/or preparing or any other processing on a food object (e.g., pet food). An RF oven may comprise apparatus 100 described above in reference to FIG. 1 or FIG. 4 and may employ any method described above, for example method 500, for applying EM energy.

In accordance with some embodiments, RF energy may be applied to the energy application zone (step 620). The EM energy may include RF energy or may consist of RF energy. Any suitable method for applying EM energy to an object (e.g., process 500 described in reference to FIG. 5 may be used to apply RF energy to an object). The present invention is not limited to the method described in reference to FIG. 5, within the scope of the invention, alternative methods might be used for applying EM energy as would be understood by a person of ordinary skill in the art, reading this disclosure.

In some embodiments, RF energy e.g., may be applied uniformly in the energy application zone. In some embodiments, RF energy may be applied in a non-uniform manner in the energy application zone. In some cases, it may be desirable to apply more energy (by weight or mass for example) to a first region of the pet food than the amount of energy applied to a second region of the pet food, for example when wishing to obtain a marbled-look of real meat. In other embodiments, a different amount of energy may be applied to the different ingredients of the mixture, for example a first amount of energy may be applied to the meat ingredients while a second amount of energy may be delivered to the other ingredients.

In some embodiments, the mixture may be shaped to a desired shape (step 630). Optionally, the mixture is shaped to a desired shape after reaching a pre-defined temperature. The mixture may be shaped to the form of biscuits, kibbles, meat-balls, patties, pellets, slices etc. The mixture may be shaped by: extrusion, pelletting, tabletting, aggregation followed by tumble coating, etc.

In some embodiments, the shaped mixture may be dried by applying RF energy to energy application zone. In some embodiments, the energy application zone may be an RF energy drying apparatus (e.g., drying oven or drying cabinet) (step 640). Any method for applying EM energy to an object as described above may be employed. In some embodiments, the cooked mixture is allowed to cool for several hours which may stabilize the moisture content of the mixture and/or may facilitate achieving an even distribution of water activity in the mixture. This process is sometimes referred to as tempering.

In some embodiments, a sterilization step (not illustrated) may be performed by applying EM energy. For example, when preparing canned or dried pet food, the food may be heated to a predefined temperature, by applying EM energy. The predefined temperature may be in the range of 90° C.-130° C. (e.g., 120° C.). The containers (e.g., cans) may be made at least partially with a conductive material. In some embodiments, RF energy is not applied to the conductive part of the can, for example, RF energy at frequencies that may be dissipated in the conductive part may not be applied.

Sterilization and Pasteurization

Some aspects of the present disclosure is to provide a method of sterilizing, sanitizing, and/or pasteurizing object(s) using RF energy, optionally in an RF sterilizer or RF pasteurizer. RF sterilizer or RF pasteurizer according to some embodiments are any apparatuses that use RF energy to reduce the amounts of bacteria in an object.

In some embodiments of the present invention, at least one object to be sterilized may be placed in an energy application zone and RF energy may be applied to the object to cause a rise of temperature in at least a portion of the object, to the desired sterilizing temperature, for example using method 500 disclosed at FIG. 5. The at least portion of the object may be held at this temperature for the time necessary to reduce the amount of undesired microorganisms and bacteria. Some embodiments may require substantially even temperature at least on the surface of the object.

In some embodiments of the present invention, the object to be sterilized is relatively dry. A relatively dry object may be an object having less than 20 weight % moisture, for example: dry food objects, dry laundry and fabrics, metals, ceramics, polymers and their composites.

In some embodiments, the object(s) to be sterilized may comprise of metal materials or may be coated with metal materials.

For example, devices or utensils comprising metals or coated by metals may be surface heated (in the metal portions) due to, for example, limited ability of metals to absorb RF energy. Nevertheless, as metal conducts heat efficiently, heat from the surface may penetrate into the bulk of the object (or at least of the metal parts). RF waves in the range of 100 MHz-100 GHz penetrate only a few microns or even less than a micron to the outer surface of the metals. This penetration may result in ohmic heating due to electric currents on the surface of the metals. However since sterilization is normally required only at the surface of the object, rise in temperature of the surface of a metallic device may result in sufficient sterilization of the heated area.

In some embodiments, the object(s) to be sterilized are comprised of dielectric materials such as polymers or ceramics. Dielectric materials may be heated up volumetrically (i.e., throughout most of all of their volume) when exposed to RF heating.

In some embodiments, the object to be sterilized may be coated with a dielectric material (or having a coating with dielectric particles therein). Such coating may be used to sterilize and/or otherwise heat the surface of the object one time or more.

In some embodiments of the present disclosure, textiles and fabrics may also be sterilized by RF heating, such as wet fabrics after washing. It is known that natural fibers like cotton may have an ability to absorb RF energy even in dry state, thus, alternatively or additionally, dry fabrics may be sterilized, for example, prior to the use. In some exemplary embodiments, clothes are at (or near) a site of use (e.g., hospital or operation room or even a kitchen) in a clean state (optionally—dry) but not sterilized, and shortly before use, may be irradiated in an RF oven to reduce bacteria counts or sterilize the material. The ability to sterilize dry items safely and efficiently may save the need to store sterilized items.

In some embodiments, the object(s) to be sterilized may comprise food items. As non-limiting examples, sterilization may be done to moisture containing food items such as meats or to dried foods (such as dried herbs and dried fruits). Dried foods may be food items having moisture content lower than 50% or 30%, 20%, 10%, or 6% in the food portion of the item.

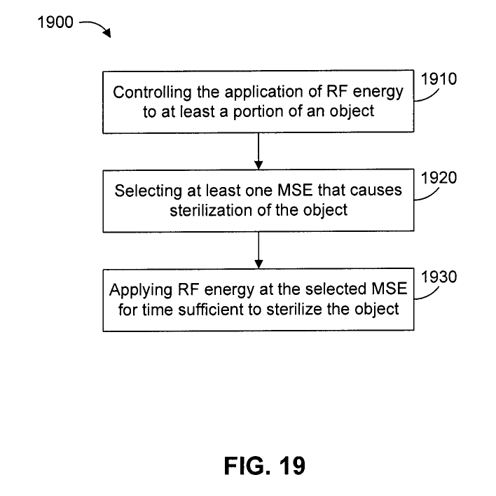

Reference is now made to method 1900, presented in FIG. 19, for applying RF to sterilized or pasteurized an object using RF energy, optionally in an RF sterilizer or RF pasteurizer. RF energy application may be controlled to at least a proton of the object, in step 1910, for example using steps 510-540 of method 500. At least one MSE may be selected in step 1920, for example according to a value indicative of RF energy absorbable in the object, determined at step 530. RF energy may be applied at the selected MSE, for a time sufficient to sterilize or pasteurize the object.

### EXAMPLES

In the following paragraphs, examples of several possible applications of the principles of the present disclosure are given, in the context of a system for selectively heating portions of an object to sterilize it.

### Example 1

### Sterilization of Herbs

Fresh parsley, with no added water, was dehydrated using RF oven. Conventional dehydration of herbs is done in convection oven heated to 40° C., to avoid over cooking and maintain the aroma and flavor of the herbs. At 40° C. many microorganisms and bacteria reproduce efficiently, thus dried herbs are known to contain large amounts of undesired bacteria, including bacteria that were present before dehydration and those that accumulated during drying.

Applying RF energy to dry fresh herbs resulted in dramatic reduction in bacteria population. 12 samples of 20 g of fresh parsley were assayed for the presence of the bacteria, and the results are shown in table 1. Six samples (group I) were soiled by immersion in a culture comprising a known concentration of Salmonella for 15 minutes and six samples (group II) were assayed without soiling. Three samples from each of group were irradiated, before being assayed, in an RF oven, having a frequency range of 800-1000 MHz, 700 Watts and two antennas for 15 min, with atmosphere heated to 40° C. (by convection) and the rest were maintained at room temperature until specimen collection. After irradiation of the relevant samples, specimens were taken from every sample and colony forming units (CFU) were counted after 72 hr incubation at 37° C. on a selective Salmonella growth medium.

During operation, a controller (e.g., controller 101) may determine a value indicative of EM energy absorbable in the object (e.g., the dissipation ratio) at different MSEs in the oven, and applied more energy over MSEs that had lower value. In some embodiments, the product of the applied energy and the dissipation ratio is substantially constant, at least across some of the MSEs. In this example, the sole difference between MSEs was the frequency of the applied MSE.

TABLE 1

| | | Average |
|---|---|---|
| group | RF treatment | Log[CFU/g] |
| Soiled | no | 7.6 |
| | yes | 5.7 |
| Un-soiled | no | 6.1 |
| | yes | 2.9 |

The parsley samples that were dehydrated by RF had a lower bacteria count in both the soiled and un-soiled samples. Such parsley is expected to maintain the flavor and aroma at least similar to parsley dehydrated by conventional means.

### Example 2

### Sterilization of Food

Mincemeat was cooked in the RF oven described in Example 1, conventionally or maintained raw, and the general amount of bacteria was compared. Five samples of 170 g of mincemeat having 20% fat were assayed. Three of the samples were soiled with 2 ml/100 g a single E-coli culture. Two of the samples (one soiled, one not soiled) were cooked in a conventional convection oven at 250° C. for 27 min to reach a core temperature of 70-80° C. Two other samples (one soiled, one not soiled) were cooked in the RF oven, preheated to 250° C. for 7 min, resulting in a similar core temperature of 70-80° C. One unsoiled sample was maintained in raw state at room temperature until all other samples were cooked. After cooking, specimens were taken from each sample counted by isolation on petri dishes as known in the art (72 hour incubation at 37° C.). Table 2 summarizes the bacteria counts.

TABLE 1

| sample | treatment | Average Log[CFU/g] | STDV |
|---|---|---|---|
| Soiled | No | 8.4 | 0.1 |
| | Conventional cooking | 2.7 | 0.8 |
| | RF cooking | 0.0 | 0.0 |
| Un-soiled | Conventional cooking | 4.0 | 0.2 |
| | RF cooking | 0.0 | 0.0 |

The two samples that were treated and cooked in the RF oven were completely sterilized. No bacteria cultures were found in those samples.

The experiments of Tables 1 and 2 were conducted under heated air to equate the environment with that of the conventional processes of drying and cooking respectively. However, similar results are believed to be obtainable also when the air was kept at room temperature (about 20-25° C.).

In the foregoing Description of Exemplary Embodiments, various features are grouped together in a single embodiment for purposes of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate embodiment of the invention.

Moreover, it will be apparent to those skilled in the art from consideration of the specification and practice of the present disclosure that various modifications and variations can be made to the disclosed systems and methods without departing from the scope of the invention, as claimed. For example, one or more steps of a method and/or one or more components of an apparatus or a device may be omitted, changed, or substituted without departing from the scope of the invention. Thus, it is intended that the specification and examples be considered as exemplary only, with a true scope of the present disclosure being indicated by the following claims and their equivalents.

What is claimed is:

1. An apparatus for applying radio frequency (RF) energy to an object in an energy application zone via at least one radiating element, wherein the apparatus is selected from a group consisting of: sterilizers, pasteurizers, drying cabinets, sintering furnaces, curing furnaces, soil remediation apparatuses, smelting furnaces, melting furnaces and plasma generators, the apparatus comprising:
at least one processor configured to:
determine a value indicative of energy absorbable by the object at least one of or each of a plurality of MSEs; and
cause RF energy to be supplied to the at least one radiating element in at least a subset of the plurality of MSEs, wherein energy supplied to the at least one radiating element at each of the subset of MSEs is a function of the value indicative of energy absorbable at each MSE.

2. The apparatus of claim 1, wherein the object is selected from a group consisting of:
pressed powders, metallic powders, ceramic powders, MMCs, pet food, metallic ores, parts to be sterilized, plasma to be generated, soil, curable polymers and dryable objects.

3. The apparatus of claim 1, further comprising a source of electromagnetic (EM) energy for supplying the RF energy to the at least one radiating element.

4. The apparatus of claim 1, further comprising at least one radiating element.

5. The apparatus of claim 1, further including a cavity, wherein the energy application zone is within the cavity.

6. The apparatus of claim 1, further comprising a system for applying a protective atmosphere to the apparatus.

7. The apparatus of claim 1, further comprising a conveyor configured to convey object to the apparatus.

8. The apparatus of claim 1, further comprising at least one sensor configured to monitor a temperature of the object.

9. The apparatus of claim 8, wherein the processor is further configured to adjust the application of RF energy based on the monitored temperature.

10. The apparatus of claim 1, further comprising at least one sensor configured to monitor a moisture level of the object.

11. The apparatus of claim 10, wherein the processor is further configured to adjust the application of RF energy based to the monitored moisture.

12. The apparatus of claim 1, further comprising at least one sensor configured to monitor contamination in the object.

13. The apparatus of claim 12, wherein the processor is further configured to adjust the application of RF energy based to the monitored contamination.

14. The apparatus of claim 1, further comprising a convection heating system.

15. The apparatus of claim 14, wherein the processor is further configured to control the convection heating system.

16. The apparatus of claim 1, wherein the at least one processor is further configured to cause the at least radiating element to apply energy to the object in an amount sufficient to heat at least a portion of the object.

17. The apparatus of claim 1, wherein the at least one processor is further configured to cause substantially uniform energy dissipation in at least a selected portion of the object at a plurality of locations of the object in the energy application zone.

18. The apparatus of claim 1, wherein the at least one processor is configured to cause substantially uniform energy dissipation in the object at a plurality of locations of the object in the zone.

19. The apparatus of claim 1, wherein the value indicative of energy absorbable at each MSE is a dissipation ratio at the corresponding MSE.

20. An apparatus for applying electromagnetic energy (EM) energy to an object in an energy application zone via at least one radiating element, wherein the apparatus is selected from a group consisting of: sterilizers, pasteurizers, drying cabinets, sintering furnaces, curing furnaces, soil remediation apparatuses, smelting furnaces, melting furnaces and plasma generators, the apparatus comprising:
at least one processor configured to:
determine a value indicative of energy absorbable by the object at least one of or each of a plurality of MSEs, and
cause energy to be supplied to the at least one radiating element in at least a subset of the plurality of MSEs, wherein the energy supplied to the at least one radiating element at each of the subset of MSEs is inversely related to the value indicative of energy absorbable at each MSE.

21. An apparatus for applying electromagnetic energy (EM) energy to an object in an energy application zone via at least one radiating element, wherein the apparatus is selected from a group consisting of: sterilizers, pasteurizers, drying cabinets, sintering furnaces, curing furnaces, soil remediation apparatuses, smelting furnaces, melting furnaces and plasma generators, the apparatus comprising:
at least one processor configured to:
determine a desired energy absorption amount at least one of or each of a plurality of MSEs; and
adjust energy supplied to the at least one radiating element at each of the plurality of MSEs in order to target the desired energy absorption amount.

22. A method for applying electromagnetic (EM) energy to an object, wherein the object is selected from a group consisting of: sterilized, pasteurized, or other pet food polymer, pressed powder, soil, metallic ores, metal, and gas, the method comprising:
controlling a source of electromagnetic EM energy in order to supply EM energy at a plurality of MSEs to at least one radiating element;
determining a value indicative of energy absorbable by the object at each of the plurality of MSEs; and
adjusting an amount of EM energy applied at each of the plurality of MSEs based on the value indicative of energy absorbable at each MSE to at least one of:
cook the sterilized or pasteurized pet food, dry the pet food, cure the polymer, sinter the pressed powder, remediate the soil, smelt the metallic ore, melt the metal, or ionize the gas.

23. A method of sterilizing at least one portion of an object using radiofrequency (RF) energy comprising:
controlling application of RF energy to the at least one portion object;
selecting at least one modulation space element (MSE) that causes at least one portion of the object to receive energy sufficient to sterilize the portion of the object; and
applying energy at the selected MSE space element to the object for a time sufficient to sterilize the portion of the object.

24. The method according to claim 23, wherein the object is dry.

25. The method according to claim 23, wherein the applying energy at the selected MSE heats the portion of the object to a desired sterilizing temperature.

26. The method according to claim 23, wherein the object is chosen from food items, food utensils, fabrics, and medical devices.

27. The method according to claim 26, wherein the object is chosen from food items having a moisture content less than 50 wt %, relative to the total weight of the at least one item.

28. The method according to claim 26, wherein the object comprises metal.

29. The method according to claim 26, wherein the object comprises at least one dielectric material.

30. The method according to claim 29, wherein the at least one dielectric material is a coating.

31. The method according to claim 26, further comprising:
determining a value indicative of RF energy absorbable in the object at a plurality of MSEs; and
applying more energy over MSEs of the plurality of MSEs that are associated with lower values of the value indicative of RF energy absorbable than over MSE, of the plurality of SEs associated with higher values of the value indicative of RF energy absorbable.
